(19) United States
(12) Patent Application Publication
(10) Pub. No.: US 2004/0092807 A1
(43) Pub. Date: May 13, 2004

(54) System and method for cancer detection

(76) Inventors: Amos Breskin, Nes Ziona (IL); Rachel Chechik, Moshav Beit Hanan (IL); Sana Shilstein, Rehovot (IL); David Vartsky, Rehovot (IL)

Correspondence Address: G.E. EHRLICH (1995) LTD., c/o ANTHONY CASTORINA, SUITE 207, 2001 JEFERSON DAVIS HIGHWAY, ARLINGTON, VA, 22202, US

(21) Appl. No.: 10/397,837

(22) Filed: Mar. 27, 2003

Related U.S. Application Data
(60) Provisional application No. 60/424,317, filed on Nov. 7, 2002.

(51) Int. Cl. A61B 5/05

(52) U.S. Cl. 600/407

(57) ABSTRACT

Apparatus for non-invasive in vivo detection of a chemical element in the prostate of a subject, comprising: (a) a probe adapted for being inserted into at least one of the rectum or the urethra of the subject; (b) an irradiation system capable of exciting the chemical element to emit radiation to form emitted radiation; (c) a radiation detector located within the probe, wherein the radiation detector is capable of detecting the emitted radiation and wherein the radiation detector is suitable for mapping the emitted radiation; and (d) a signal recording, processing and displaying system for mapping the level of tie chemical element in the prostate of the subject at a plurality of different points in the prostate according to the mapping of the emitted radiation. In one embodiment, the irradiation system is capable of delivering exciting radiation through the probe to the prostate; in another embodiment the emitted radiation comprises fluorescent X-ray radiation.

This is a continuation in part of U.S. Provisional Patent Application No. 60/424,317, filed Nov. 7, 2002 which claims the benefit of priority from PCT Patent Application No. IL01/00902 filed Sep. 25, 2001 and IL Patent Application No. 138756 filed Sep. 28, 2000, which are hereby incorporated by reference.

### FIELD AND BACKGROUND OF THE INVENTION

The present invention relates to in vivo detection of chemical elements in the prostate and, more particularly, to an apparatus for and method of detecting and staging of prostate cancer by in vivo determination and mapping of zinc in the prostate.

### BACKGROUND OF THE INVENTION

Carcinoma of the prostate is the most common form of cancer in men. The methods commonly used today for detection of prostate cancer are digital rectal examination (DRE), transrectal ultrasound (TRUS) and prostate-specific-antigen (PSA) determination. It is recognized that none of the above methods is sufficiently accurate, hence a prostate carcinoma diagnosis is often based on a combination of two or more examinations.

PSA testing is the most common assay used in diagnosis of prostate cancer and particularly in screening. In normal men, only minute amounts of PSA circulate in the serum. Elevated PSA levels in blood occur in association with localized as well as advanced prostate cancer. In most laboratories a serum level of 4 ng/ml is used as a cut-off point between normal and abnormal.

However, elevations in PSA occur not only in cancer cases but also in some non-neoplastic conditions, such as nodular hyperplasia and prostatitis. There is a considerable overlap in levels of serum PSA between that found in such conditions and that found in prostate cancer patients. For instance, 25 to 30% of men with nodular hyperplasia and 80% with histologically documented cancer have PSA serum level greater than 4 ng/ml.

A number of recent studies and surveys for assessing the effectiveness of this method in prostate cancer diagnosis appear in the literature. A 1999 study of Luboldt et al. [Luboldt H J, Altwein J E, Bichler K H, Czaja D, Husing J, Fomara P, Jockel H H, Lubben G, Schalkhauser K, Weissbach L, Wirth M and Rubben H: Urologe-Ausgabe A38:114-123, 1999] encompassed 12,542 men with a mean age of 62±7.5 who were tested using DRE findings and PSA. Of theses, 2343 had suspected DRE or PSA level exceeding 4 ng/ml. Biopsies taken from 744 of the men in the latter category revealed only 157 (20%) cases of confirmed prostate cancer.

Another 1999 study of Tomblom et al. [Tomblom M, Norming U, Adolfsson J, Becker C, Abrahamsson P A, Lilja H and Gustafsson O. Urology 945-950, 1999] tested 1748 men, of whom 367 underwent biopsies due to abnormal findings on either DRE, TRUS or had PSA levels exceeding 10 ng/ml. This led to the diagnosis of 64 cases of prostate cancer (17.4%). Moreover, this study revealed that 14% of patients with prostate cancer had PSA levels inferior to 3 ng/ml.

In view of such overlap, several refinements in the estimation and interpretation of PSA values have been proposed, such as PSA density (ratio of PSA level to the volume of the prostate gland), PSA velocity (rate of increase in PSA level with time), age-specific reference values and the ratio of free-to-total PSA in the serum; denoted as percent free PSA, (% FPSA). While many of these parameters are still under investigation, the characterization via % FPSA appears to have a particular value for distinguishing prostatic cancer from non-neoplastic conditions. Catalona et al [Catalona, W. J., Clinical utility of measurements of free and total prostate-specific antigen (PSA): A review, Prostate 7:64, 1996; Catalona, W. J., Partin, A. W., Finlay, J. A., Chan, D. W., Rittenhouse, H. G., Wolfert, R. L., and Woodrum, D. L., Use of percentage of free prostate specific antigen to identify men at high risk of prostate cancer when PSA levels are 2.51 to 4 ng/ml and digital examination is not suspicious for prostate cancer: An alternative model, Urology, 54:220-224, 1999] have proposed a model using % FPSA for detecting prostate cancer in the particular group of patients having PSA values between 2.51 and 4 ng/ml and DRE with no pathological findings. This model recommends biopsy for 10% to 36% of the men in this population and predicts a cancer detection rate of 30% to 54%. Tomblom's study indicated that combination of PSA levels inferior to 3 ng/ml and % FPSA exceeding 18% defines a large portion of the population as running a very low risk of prostate cancer, however the authors warn that the risk of contracting prostate cancer is not negligible in men with PSA inferior to 3 ng/ml who exhibit a % FPSA of 18% or less.

Although it appears that % FPSA has merit for discriminating between benign and malignant disease in cases where the total PSA is in the “gray zone” of 4 to 10 ng/ml, pending a situation where the above-mentioned refinements are better established, serum PSA by itself cannot be used for detection of early cancer and needs to be combined with other diagnostic indicators.

In addition to the above-mentioned deficiencies, the existing methods do not provide sufficient information about the stage of the disease, namely the tumor dimension and the level of cancer proliferation. Moreover, when cancer is suspected a biopsy procedure is usually performed. The lack of precise information as to the tumor localization renders the biopsy procedure inefficient.

It is well established that a normal human prostate gland contains high levels of zinc (Zn). Although reported values vary considerably, whole prostate preparations contain Zn concentrations of about 150 μg/g wet weight, which is about 2-5 times greater than Zn content of most other tissues. Zinc is not uniformly distributed throughout the prostate and, as demonstrated by Gyorkey et al. [Gyorkey, F., Min K. W., Huff, J. A and Gyorkey, P., Zinc and magnesium in human prostate gland: normal hyperplastic and neoplastic, Cancer, 27:1348, 1967], the highest Zn content (211 μ/g wet weight) is found in the lateral lobe of the peripheral zone. Numerous in vitro studies [Gyorkey et al., ibid; Lahtonen, R., Zinc and cadmium concentrations in whole tissue and separated epithelium and stroma from human benign prostatic hypertrophic glands; Prostate, 6:177, 1985; Gonic, P., Oberleas D., Knechtges T. and Prasad, A. S., Atomic absorption determination of zinc in the prostate; Invest. Urol., 6:345, 1969; Dhar, N. K., Goel, T. C., Dube, P. C., Chowdury, A. R. and Kar, A. B., Distribution and concentration of zinc in the subcellular fractions of benign hyperplastic and malignant neoplastic human prostate, Exp. Mol. Pathol., 19:139, 1973; Habib, F. K., Mason, M. K., Smith, P. H., and Stitch, S. R., Cancer of the prostate: early diagnosis by zinc and hormone analysis, Br. J. Cancer 39:700, 1979; Ogunlewe, J. O. and Osegbe, D. N., Zinc and cadmium concentrations in indigenous blacks with normal, hypertrophic and malignant prostate, Cancer, 63:1388, 1989; Feustel, A., Wennrich, R., Steiniger, D. and Klauss, P., Zinc and cadmium concentration in prostatic carcinoma of different histological grading in comparison to normal prostate tissue and adenofibromyomatosis (BPH), Urol. Res. 10:301, 1982; and Zaichick, V. Y., Sviridova, T. V. and Zaichick, S. V., Zinc in the human prostate gland, normal, hyperplastic and cancerous, Int. Urol. Nephrol., 29:687-694, 1997] indicate that Zn concentration in the prostate is substantially lower in cancerous tissue compared to benign prostate hyperplasia (BPH) and normal prostate tissue. Zaichick et al. (ibid) reported dry weight Zn concentrations of 1018±124, 1142±77 and 146±10 μg/g for normal, BPH and cancerous prostate, respectively. In addition, they found that the decrease in Zn levels in cancer starts at very early stages of the disease and there is a lack of Zn level dependence on the stages of the disease and on histological cancer grading. Zinc levels are modified only in the cancerous tissue. Tissues not involved in the tumor process remain unaltered and zinc levels in visually and morphologically intact tissues are at normal levels.

It is important to note that the Zn levels in prostate cancer approach the typical levels normally associated with non-prostate tissue, which would indicate that the malignant prostate epithelial cells have lost the ability to accumulate zinc. Based on this finding, Habib et al., 1979 (ibid), suggested that the decrease in zinc was an early step in malignancy and could be used for early diagnosis of prostate cancer. It has been suggested that decreased zinc accumulation occurs in cell population prior to their histopathological identification as malignant cells and that this represents biochemical changes early in the malignant process, possibly as a premalignant stage [Cotran R. S., Kumar V. and Collins T., Robbins Pathologic Basis of Disease, W. B. Sounders Co., Sixth Edition, 1029, 1999].

The association of early decrease in Zn concentration and the appearance of prostate cancer led to the establishment of the bioenergetic theory of prostate malignancy, according to which the prostate cell becomes citrate oxidizing (instead of citrate producing) in order to meet the energy demands of the neoplastic process; this is achieved by depleting the Zn deposits in the mitochondria, allowing the m-aconitase mediated conversion of citrate to isocitrate and its subsequent oxidation in its normal metabolic pathway [Costello L C, Franklin R B. Bioenergetic theory of prostate malignancy, Prostate 25:162-166, 1994; Costello L C, Franklin R B, Liu Y, Kennedy M C, Zinc causes a shift toward citrate at equilibrium of the m-aconitase reaction of the prostate mitochondria, Journal of Inorganic Biochemistry 78:161-165, 2000].

In spite of being a proven discriminator between benign and cancerous prostate tissues, the detection of Zn levels is presently not commonly employed in medical institutes, because of the lack of appropriate apparatus and methods to perform such examination in vivo on patients.

### SUMMARY OF THE INVENTION

There is thus a widely recognized need for, and it would be highly advantageous to have, an apparatus for and method of detecting and staging of prostate cancer by in vivo determination and mapping of zinc in the prostate. Such in vivo determination may be used for a more reliable differentiation between cancerous tissue and that of benign prostate hyperplasia and normal tissue, hence reducing the required number of biopsies, with the important consequence of cost reduction in healthcare. Moreover, in vivo zinc determination and mapping may reduce the rate of false-negative diagnosis, thus minimizing the mortality from undetected prostate cancer.

The background art does not teach in vivo zinc determination and mapping of the prostate. The present invention provides a method and apparatus which can be efficiently used for many medical applications, such as, but not limited to, endoscopic diagnosis and treatment, including for the above in vivo zinc determination and mapping. The present invention also provides a method and apparatus for in-vitro examinations, e.g., of needle-biopsy samples, according to which medical diagnoses are significantly improved.

Thus, according to one aspect of the present invention there is provided an apparatus for non-invasive in vivo detection of a chemical element in the prostate of a subject, comprising: (a) a probe adapted for being inserted into at least one of the rectum or the urethra of the subject; (b) an irradiation system capable of exciting the chemical element to emit radiation to form emitted radiation; (c) a radiation detector located within the probe, wherein the radiation detector is capable of detecting the emitted radiation and wherein the radiation detector is suitable for mapping the emitted radiation; and (d) a signal recording, processing and displaying system for mapping the level of the chemical element in the prostate of the subject at a plurality of different points in the prostate according to the mapping of the emitted radiation.

According to yet another aspect of the present invention there is provided a system for diagnosing prostate cancer in the prostate of a subject, the system comprising (a) a first apparatus for determining a first parameter being a level of a chemical element in the prostate; (b) a second apparatus for determining a second parameter being indicative of prostate specific antigen (PSA) activity in the blood serum of the subject; and (c) a data processor programmed to diagnose the prostate cancer if the first parameter has a predetermined relation with respect to a first predetermined threshold and the second parameter has a predetermined relation with respect to a second predetermined threshold.

According to further features in preferred embodiments of the invention described below, the irradiation system is capable of delivering exciting radiation through the probe to the prostate.

According to still further features in the described preferred embodiments the first apparatus is operable to detect the first level of the chemical element in vivo or in vitro.

According to still further features in the described preferred embodiments the second parameter is selected from the group consisting of serum PSA level, PSA density, PSA velocity, a level of age specific PSA, and percentage of free PSA.

According to still further features in the described preferred embodiments each of the first and the second parameters may independently be either above or below its respective predetermined threshold, depending on the parameter.

According to still further features in the described preferred embodiments the first apparatus is an X-ray fluorescence-based apparatus.

According to still further features in the described preferred embodiments the second apparatus selected from the group consisting of an activation analysis-base apparatus, an atomic absorption-based apparatus, and a particle-induced X-ray emission-based apparatus.

According to still further features in the described preferred embodiments the system further comprises a biopsy device.

According to still further features in the described preferred embodiments the first apparatus comprises (i) a probe adapted for being inserted into at least one of the rectum or the urethra of the subject; (ii) an irradiation system capable of exciting the chemical element to emit radiation to form emitted radiation; and (iii) a radiation detector located within the probe, wherein the radiation detector is capable of detecting the emitted radiation and wherein the radiation detector is suitable for mapping the emitted radiation.

According to still another aspect of the present invention there is provided a system for mapping a prostate of a subject, the system comprising: (a) at least one mapping device; (b) an irradiation system capable of exciting a chemical element in the prostate to emit radiation to form emitted radiation; (c) an endoscopic probe for detecting the chemical element, wherein the endoscopic probe comprises a radiation detector capable of detecting the emitted radiation and capable of mapping the emitted radiation; and (d) a data processor for mapping the prostate according to information collected from the at least one mapping device and the endoscopic probe.

According to further features in preferred embodiments of the invention described below, the emitted radiation comprises fluorescent X-ray radiation.

According to still further features in the described preferred embodiments the irradiation system is capable of delivering exciting radiation through the probe to the prostate.

According to still further features in the described preferred embodiments the at least one mapping device is selected from the group consisting of an ultrasonic device, a magnetic-resonance-imaging device and a computer tomography device.

According to still further features in the described preferred embodiments the radiation detector comprises at least one of a high energy-resolution solid state detector and a high energy-resolution gaseous detector.

According to still further features in the described preferred embodiments the radiation detector comprises at least one of a scanning detector, a position-sensitive detector or an array of detectors or a combination thereof.

According to still further features in the described preferred embodiments the high energy-resolution gaseous detector is selected from the group consisting of a gas proportional detector and gas scintillation detector.

According to still further features in the described preferred embodiments the high energy-resolution solid state detector is selected from the group consisting of Silicon radiation detector, Germanium radiation detector, Silicon-Lithium-drifted radiation detector, Germanium-Lithium-drifted radiation detector, Mercury Iodide radiation detector and Cadmium-Zinc Telluride radiation detector.

According to still further features in the described preferred embodiments the solid-state radiation detector is selected from the group consisting of a PIN diode, a surface barrier diode, a drift diode, a micro-strip detector, a drift chamber, a multi-pixel detector and a multi-strip detector.

According to still further features in the described preferred embodiments the irradiation system comprises a scanning irradiation system.

According to still further features in the described preferred embodiments the radiation detector is capable of detecting radiation from a plurality of predetermined angles so as to allow the signal recording, processing and displaying system to map the level of the chemical element at the plurality of different points.

According to still further features in the described preferred embodiments the apparatus further comprising an arrangement of radiation detectors for detecting radiation from a plurality of predetermined angles so as to allow the signal recording, processing and displaying system to map the level of the chemical element at the plurality of different points.

According to still further features in the described preferred embodiments the chemical element comprises zinc, wherein the radiation detector and the irradiation system are suitable for measuring the level of zinc, and wherein the signal recording, processing and displaying system maps the level of zinc to detect a possible cancer in at least a portion of the prostate.

According to still further features in the described preferred embodiments the chemical element to be detected emits characteristic fluorescent X-rays according to an identity of the chemical element, and wherein an intensity of the characteristic fluorescent X-rays correlates to a concentration of the chemical element, such that the radiation detector is adapted to detect at least one chemical element according to the characteristic fluorescent X-rays and to measure the intensity.

According to still further features in the described preferred embodiments the radiation detector is suitable for measuring the level of at least one radioactive substance introduced into the prostate.

According to still further features in the described preferred embodiments the signal recording, processing and displaying system maps a boundary of possible cancer in the prostate.

According to still further features in the described preferred embodiments the signal recording, processing and displaying system maps the boundary according to a distribution of the chemical element in at least a region of the prostate being examined.

According to still further features in the described preferred embodiments the boundary is at least partially determined according to a distribution of different concentrations of the chemical element within at least the region.

According to still further features in the described preferred embodiments the distribution of the different concentrations of the chemical element is also used for staging the cancer.

According to still further features in the described preferred embodiments the apparatus further comprising at least one additional mapping device for combining with information from the signal recording, processing and displaying system for determining the boundary.

According to still further features in the described preferred embodiments the at least one additional mapping device is selected from the group consisting of a transrectal ultrasound probe and a magnetic-resonance-imaging probe.

According to still further features in the described preferred embodiments the chemical element comprises a chemical element introduced into the prostate for a specific medical procedure, and wherein the signal recording, processing and displaying system maps the level of the chemical element to perform the specific medical procedure on at least a portion of the prostate.

According to still further features in the described preferred embodiments the specific medical procedure comprises a photodynamic therapy.

According to still further features in the described preferred embodiments the chemical element is introduced in either a quantitative or a qualitative amount According to still further features in the described preferred embodiments the radiation detector detects X-ray fluorescence.

According to still further features in the described preferred embodiments the irradiation system comprises at least one of a radioactive source, an X-ray tube, a synchrotron light source, an X-ray beam guide connected to an external X-ray source or a miniature plasma X-ray generator.

According to still further features in the described preferred embodiments the irradiation system is coupled to a monochromatizing element so as to provide a radiation with a substantially accurate energy.

According to still further features in the described preferred embodiments the monochromatizing element is selected from the group consisting of a crystal monochromator and a plurality of different absorbing films each characterized by a different absorption coefficient.

According to still further features in the described preferred embodiments the apparatus further comprising a biopsy device.

According to still further features in the described preferred embodiments the apparatus further comprising a device for injection of a drug or a contrast agent.

According to still further features in the described preferred embodiments the apparatus further comprising a device for illumination of the prostate with light.

According to still further features in the described preferred embodiments the apparatus further comprising a normalizer for normalizing measurement of the emitted radiation according to a normalizing measurement of a reference element.

According to still further features in the described preferred embodiments the radiation detector is characterized by geometry selected from the group consisting of planar geometry, spherical geometry, cylindrical geometry and an irregular geometry.

According to still further features in the described preferred embodiments the apparatus further comprising an X-ray optical system, located within the probe, wherein the X-ray optical system is selected so as to collimate and/or focus radiation emitted by the irradiation system and/or radiation emitted by the chemical element.

According to still further features in the described preferred embodiments the X-ray optical system comprises a focusing element for focusing the radiation emitted by the irradiation system.

According to still further features in the described preferred embodiments the focusing element is selected from the group consisting of a capillary optical device and an aperture.

According to still further features in the described preferred embodiments the X-ray optical system comprises a collimating element for collimating the radiation emitted by the irradiation system.

According to still further features in the described preferred embodiments X-ray optical system comprises a capillary X-ray optics for focusing and collimating the radiation emitted by the irradiation system.

According to still ether features in the described preferred embodiments the X-ray optical system comprises a collimator for collimating the radiation emitted by the chemical element into the radiation detector.

According to still further features in the described preferred embodiments the collimator is characterized by geometry selected from the group consisting of planar geometry, spherical geometry, cylindrical geometry and an irregular geometry.

According to still further features in the described preferred embodiments the collimator is made of a substrate having a plurality of predetermined radiation paths, wherein the plurality of predetermined radiation paths is selected from the group consisting of radiation paths directing radiation emitted from the chemical element in a single location to a plurality of locations on the radiation detector, radiation paths directing the radiation emitted from the chemical element in a plurality of locations to a plurality of locations on the radiation detector, and radiation paths directing the radiation emitted from the chemical element in a plurality of locations to a plurality of detector-elements.

According to still further features in the described preferred embodiments, each of the plurality of predetermined radiation paths is selected from the group consisting of a thin aperture, a thin capillary and an X-ray optical element.

According to still further features in the described preferred embodiments the radiation detector is capable of discriminating between radiation emitted by the chemical element being present in the prostate and radiation emitted by chemical elements being present in tissues surrounding the prostate, thereby to map the prostate.

According to still further features in the described preferred embodiments the apparatus further comprising a collimator for collimating the emitted radiation in a manner that radiation emitted by chemical elements being present in tissues other than tissues of the prostate is absorbed by the collimator.

According to still further features in the described preferred embodiments the radiation detector is capable of simultaneously detecting the emitted radiation from a plurality of depth inside the prostate.

According to still further features in the described preferred embodiments the apparatus further comprising an arrangement of radiation detectors and a collimator, wherein the collimator is capable of collimating radiation emitted from different depths inside the prostate into different locations of a radiation detector or different radiation detectors.

According to still further features in the described preferred embodiments the apparatus further comprising electronic circuitry, adapted for being located within the probe, wherein the electronic circuitry is designed and constructed for transmitting signals from the radiation detector to the signal recording, processing and displaying system.

According to still further features in the described preferred embodiments the apparatus further comprising a thermoelectric cooling system inside the probe for cooling the detector, as to obtain the best possible energy resolution.

According to still further features in the described preferred embodiments the apparatus further comprising a transrectal ultrasound probe.

According to an additional aspect of the present invention there is provided a method of non-invasive in vivo detection of a chemical element in the prostate of a subject, comprising: endoscopically inserting a probe into the subject; irradiating the prostate with the probe by exciting radiation thereby exciting the chemical element to emit radiation to form emitted radiation; detecting and mapping the emitted radiation with the probe; and mapping the level of the chemical element in the prostate of the subject at a plurality of different points in the prostate according to the mapping of the emitted radiation.

According to yet an additional aspect of the present invention there is provided a method of diagnosing prostate cancer in the prostate of a subject, the method comprising: determining a first parameter being a level of a chemical element in the prostate; determining a second parameter being indicative of prostate specific antigen (PSA) activity in the blood serum of the subject; and wherein the prostate cancer is diagnosed if the first parameter has a predetermined relation with respect to a first predetermined threshold and the second parameter has a predetermined relation with respect to a second predetermined threshold.

According to further features in preferred embodiments of the invention described below, the determining the level of the chemical element is done in vivo or in vitro.

According to still further features in the described preferred embodiments each of the first and the second parameters may independently be either above or below its respective predetermined threshold, depending on the parameter.

According to still further features in the described preferred embodiments second parameter is selected from the group consisting of serum PSA level, PSA density, PSA velocity, a level of age specific PSA, and percentage of free PSA.

According to still further features in the described preferred embodiments determining the level of the chemical element is by X-ray fluorescence.

According to still further features in the described preferred embodiments determining the level of the chemical element is affected by a procedure selected from the group consisting of an activation analysis, an atomic absorption a particle-induced X-ray emission.

According to still an additional aspect of the present invention there is provided a method of mapping a prostate of a subject, the method comprising: endoscopically inserting a probe into the subject; irradiating the prostate with the probe by exciting radiation thereby exciting the chemical element to emit radiation to form emitted radiation; detecting and mapping the emitted radiation with the probe; mapping the prostate using at least one additional mapping device; and collecting information from the at least one additional mapping device and the probe, so as to map the prostate.

According to further features in preferred embodiments of the invention described below, the probe is endoscopically inserted into the rectum or the urethra of the subject.

According to still further features in the described preferred embodiments the detecting the emitted radiation is by a radiation detector which comprises at least one of a scanning detector, a position-sensitive detector or an array of detectors or a combination thereof.

According to still further features in the described preferred embodiments the radiation detector comprises at least one of a high energy-resolution solid state detector and a high energy-resolution gaseous detector.

According to still further features in the described preferred embodiments the high energy-resolution gaseous detector is selected from the group consisting of a gas proportional detector and gas scintillation detector.

According to still further features in the described preferred embodiments the irradiating comprises scanning the prostate so as to excite the chemical element to emit the fluorescent X-ray radiation from a plurality of predetermined angles.

According to still further features in the described preferred embodiments the radiation detector is a scanning detector or a position-sensitive detector.

According to still further features in the described preferred embodiments the detecting the emitted radiation is by scanning the prostate so as to detect the emitted radiation from a plurality of predetermined angles.

According to still further features in the described preferred embodiments the detecting the emitted radiation is by an arrangement of radiation detectors arranged so as to detect the emitted radiation from a plurality of predetermined angles.

According to still further features in the described preferred embodiments the chemical element comprises zinc, and wherein the level of zinc is used for detecting a possible cancer in at least a portion of the prostate.

According to still further features in the described preferred embodiments the method further comprising introducing at least one radioactive substance into the prostate and measuring the level of the at least one radioactive substance in the prostate.

According to still further features in the described preferred embodiments the method further comprising mapping a boundary of the possible cancer in the prostate.

According to still further features in the described preferred embodiments the chemical element comprises a chemical element introduced into the prostate for a specific medical procedure, and wherein the mapping the level of the chemical element is used for performing the specific medical procedure on at least a portion of the prostate.

According to still further features in the described preferred embodiments the specific medical procedure comprises a photodynamic therapy.

According to still further features in the described preferred embodiments the chemical element to be detected comprises one or more of Zn, Fe, Ca, Br, or Pd.

According to still further features of the preferred embodiments the concentration of a given chemical element is normalized to an amount of Compton scattered radiation of the incident radiation.

According to still further features in the described preferred embodiments the method further comprising using the probe for performing a biopsy procedure.

According to still further features in the described preferred embodiments the method further comprising using the probe for injection of a drug or a contrast agent into the prostate.

According to still further features in the described preferred embodiments the method further comprising using the probe for illuminating the prostate with light.

According to still further features in the described preferred embodiments the method further comprising a normalizing measurement of the emitted radiation according to a normalizing measurement of a reference element.

According to still further features in the described preferred embodiments the method further comprising collimating and focusing the exciting radiation and the emitted radiation.

According to still further features in the described preferred embodiments the method further comprising imaging the prostate using a transrectal ultrasound probe.

The present invention successfully addresses the shortcomings of the presently known configurations by providing a method and apparatus for non-invasive in vivo detection of a chemical element in the prostate.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of an apparatus for non-invasive in vivo detection of a chemical element in the prostate of a subject, according to a preferred embodiment of the present invention;

FIG. 2a is a schematic illustration a probe of the apparatus, according to a preferred embodiment of the present invention;

FIG. 2b is a schematic illustration of the probe of the apparatus, having a collimator capable of simultaneous detection of radiation from different depths, according to a preferred embodiment of the present invention.

FIGS. 2c-d are schematic illustration planar (c) and spherical (d) geometry of a collimator of the apparatus, according to a preferred embodiment of the present invention;

FIG. 3 illustrates a preferred use of the apparatus, according to which the probe is introduced through the rectum in close proximity to the peripheral zone of the prostate;

FIG. 4a shows a flowchart of a method of non-invasive in vivo detection of a chemical element in the prostate of a subject, according to a preferred embodiment of the present invention, while FIG. 4b shows a flowchart of a method of in vitro analysis after a needle biopsy has been performed;

FIG. 5 shows an experimental arrangement for in vitro measurements of X-ray spectrum of prostate samples or phantom;

FIG. 6 shows a spectrum obtained from irradiation of a vial containing 1000 μg/g of Zn aqueous solution;

FIGS. 7-8 show X-ray fluorescence spectra obtained from prostate specimens embedded in paraffin and prepared for histological examination, diagnosed as benign prostate hyperplasia (7) and prostate cancer (8);

FIGS. 9a-b show XRF spectra of Zn content in prostate samples diagnosed as prostate cancer (a) and benign prostate hyperplasia (b);

FIG. 10 shows a graphical representation of zinc concentrations in prostate samples for benign prostate hyperplasia (BPH), prostate cancer (CAP) and CAP/BPH;

FIG. 11 shows correlation between the zinc content and the prostate-specific-antigen values, for benign prostate hyperplasia (BPH), prostate cancer (CAP) and CAP/BPH;

FIG. 12a shows an experimental system for in-depth topographic zinc determination of prostate phantom;

FIG. 12b shows the prostate phantom which comprises two flat containers filled with tissue equivalent solution containing known zinc concentrations;

FIGS. 13a-b show schemes of beams crossing inside the prostate phantom for scattering angles 90′ (a) and 150′ (b);

FIGS. 14a-b show experimental result of a response function for scanning a Cu foil for 90′ (a) and 150° (b) configurations;

FIGS. 15a-e show the results of phantoms scans for different zinc concentration ratios;

FIG. 16 shows a ratio of fluorescent intensities at depth of 2 mm to that obtained from the surface for the above scans as a function of the zinc concentration ratio;

FIG. 17 shows a ratio of the K_{α} to K_{β} intensities in the uniform phantom as a function of the depth in the phantom; and

FIG. 18 shows results of scanning measurements performed at scattering angle of 150′, for zinc concentration ratio of 4.

### DESCRIPTION OF THE PREFERRED EMBODIMENTS

The present invention is of a system, apparatus and method for non-invasive in vivo detection of a chemical element in the prostate of a subject, which can preferably be zinc. Specifically, and more preferably the present invention can be used for detecting and staging of prostate cancer by in vivo determination and mapping of zinc in the prostate. The present invention is further of a system and method for combining the information of the chemical element with information collected, for example, from prostate-specific-antigen (PSA) analysis or a mapping device, e.g., ultrasonic device and the like.

Since it is recognized that even for cases of prostate cancer, not all of the tissue is expected to be cancerous, the present invention provides an accurate and useful measurement which enables the levels of zinc to be mapped throughout the prostate, so that changes in a specific part of the prostate could be accurately detected. It would be appreciated that such mapping throughout the prostate is more likely to result in an accurate diagnosis. Moreover, as prostate mapping provides valuable information regarding cancerous and benign regions of the prostate, such mapping is important for decisions regarding surgery and/or other prostate cancer therapies.

As used herein, the terms “determining”, “determine” or “determination” interchangeably refer to qualitative determination, namely detecting the presence of a certain element in the prostate tissue, or quantitative determination, namely determination of the amount or level of an element in the tissue.

The principles and operation of exemplary apparatus and method according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present invention is primarily directed at detecting chemical element via X-ray fluorescence (XRF). For the purpose of clarity, the basic principles of XRF are described first.

XRF is an analytical method widely used for analysis of trace elements in various matrices. Biological samples such as tissues can be analyzed intact by XRF without sample processing. In XRF, the analyzed tissue may be exposed to a low radiation dose of X-rays or low energy gamma rays from an X-ray tube or an isotopic radioactive source, which as described herein are non-limiting examples of irradiation systems and/or may form a component of such a system. This radiation causes the excitation of the atoms present in the tissue, which in turn decay by emission of characteristic fluorescent X-rays. The characteristic X-rays emitted from the sample are detected and counted by a high energy-resolution detector. The intensity of these X-rays is directly proportional to the concentration of the elements inside the tissue. In the case of Zn, for example, the characteristic fluorescent X-ray energies are 8.6 and 9.6 keV. The sensitivity of the XRF method depends on the chemical element of interest and on the experimental conditions. The limits of detection are typically below 1 μg/g, e.g. 1 part per million.

Since Zn concentrations in the prostate are about 5 times lower in cancerous tissue compared to normal and benign prostate hyperplasia (BPH), an accurate in vivo mapping of Zn concentration has a significant impact on the diagnosis reliability and on preoperative staging. In vivo mapping of Zn and/or other chemical element in the prostate of a subject are addressed by the present invention by providing a method and apparatus for non-invasive in vivo detection of a chemical element in the prostate of a subject.

Before providing a further detailed description of the method and apparatus, in accordance with the present invention, attention will be given to the advantages and potential applications offered thereby.

Hence, although XRF measurements of zinc levels in prostate tissue have been shown to be efficient in differentiating between benign and malignant tumors, all background art measurements were performed on tissues which were removed from the subject at some earlier stage. The present invention successfully provides a non-invasive measurement of zinc levels in the prostate of the subject, without the need for a biopsy. The advantage of the non-invasive technique of the present invention is at least threefold.

First, as a skilled artisan would appreciate, the results may be obtained from such measurement essentially in real-time, as opposed to the biopsy where the ablated tissue is sent for further laboratory examination.

Second, it is recognized that needle-biopsy based measurements can only provide information on the status of the prostatic tissue at a limited number of selected points from which the needle-biopsy was extracted. The present invention provides a more complete and useful information on the Zn concentration levels mapped over the whole region of the prostate near the rectal wall, so that changes in a specific part of the prostate near the rectal wall could be accurately detected.

Third, as further demonstrated in the Examples section that follows, while conceiving the present invention it has been uncovered that there is a correlation between the zinc content and values of prostate-specific-antigen, which correlation has been proven to be exploitable in an accurate detection and diagnosis of prostate cancer.

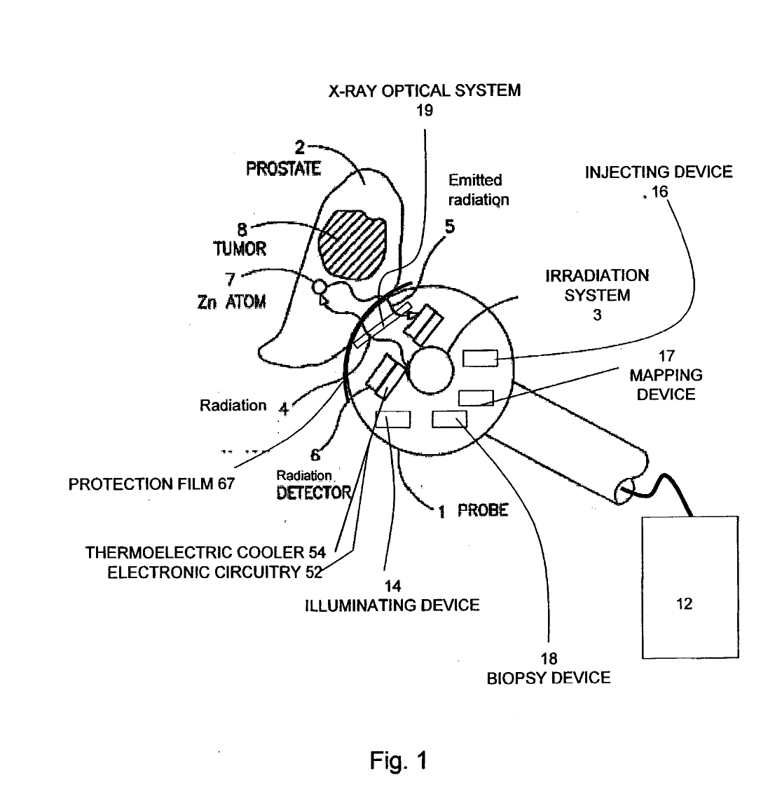

Referring now to the drawings, FIG. 1 illustrates the apparatus for non-invasive in vivo detection of a chemical element in the prostate of a subject, generally referred to herein as apparatus 10.

Hence, apparatus 10 comprises a probe 1 adapted for being inserted into at least one of the rectum or the urethra of the subject. Probe 1 is preferably flexible so as to facilitate the insertion of probe 1 into the anus or through the urethra. Additionally and preferably probe 1 including its various components as further detailed hereinafter, is size wise and geometrically compatible with the internal cavities of the subject so as to minimize discomfort of the subject during the non-invasive in vivo examination.

It is known that in most cases (about 70-80%), carcinoma of the prostate originates in the peripheral zone of the posterior lobe, which may be diagnosed by access through the rectum. For the purpose of diagnosing other (e.g., central) parts of the prostate, access is preferably through the urethra. Thus, as stated, probe 1 is preferably adapted for both transrectal and transurethral examination. One ordinarily skilled in the art would appreciate that chemical element determination may also be needed during medical operation. Thus, probe 1 is preferably designed as an interoperative probe, which can be conveniently used by the surgeon or an assistant. Alternatively, and preferably, several probes may be provided, e.g., a rectal probe a urethral probe and an interoperative probe, depending on the application for which apparatus 10 is to be used.

As used herein, the term “probe” refers to a rectal probe, a urethral probe, an interoperative probe or a probe designed for more than one medical application, as further detailed hereinabove.

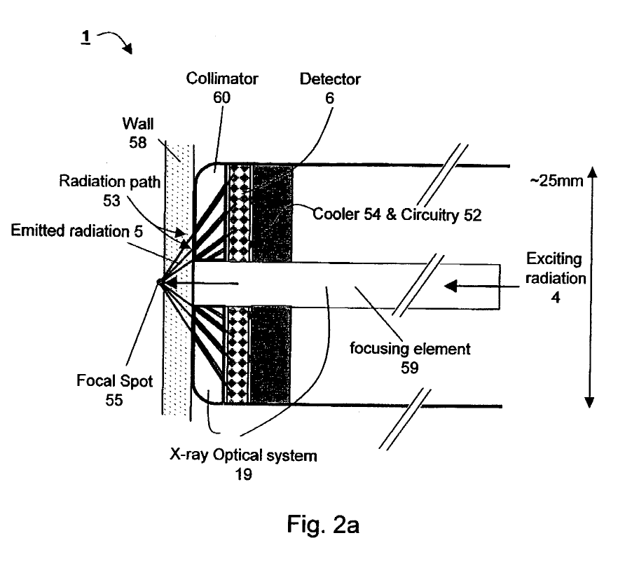

A detailed description of probe 1, according to a preferred embodiment of the present invention will be provided hereinafter (with reference to FIGS. 2a-d). Following is a general description of apparatus 10.

Thus, apparatus 10 further comprises an irradiation system 3, at least a portion of which may optionally be located within probe 1, which is capable of emitting exciting radiation 4 so as to excite a chemical element (e.g., Zn atom 7 shown in FIG. 1) to emit characteristic radiation 5 (e.g., fluorescent X-ray radiation). Specifically, irradiation system 3 emits radiation 4 in a desired energy, flux and direction so as to impinge on the tissue of prostate 2. This radiation causes the excitation of chemical element 7, which in turn decays by emission of emitted radiation 5.

According to a preferred embodiment of the present invention irradiation system 3 may be, for example, a conventional radioactive source such as, but not limited to, a ¹⁰⁹Cd source, an X-ray tube such as, but not limited to, a miniature X-ray tube, a synchrotron light source, an X-ray beam guide connected to an external X-ray source, a miniature plasma X-ray generator and the like.

The energy of the incident exciting photons emitted from irradiation system 3 is dictated by the energy behavior of the cross-section for the excitation of a given element and by the background produced by scattering of the incident radiation on the large mass of surrounding tissue. Preferably, the energy of the incident radiation is selected to optimize the measurement. Specifically, the energy is sufficiently high so as to reduce this background, but not too high so as not to reduce the cross-section for the excitation. For example, if the chemical element is zinc, the incident energy must be just above the K-edge energy of Zn (9.66 keV).

Two additional factors are also preferably considered, namely, the ability of the incident radiation to penetrate inside the prostate through the rectal wall and the background that it produces in the spectral region of the characteristic radiation of Zn (8.6 and 9.6 keV). Both factors dictate a preferred incident energy higher than 9.66 keV and an optimal energy must be found.

Hence, for example, when a monoenergetic synchrotron radiation is used as an incident radiation the optimal energy is preferably about 13 keV for a 3 mm thick rectal wall. When a filtered X-ray tube is used the energy depends on the anode material and the filtration of the continuous bremsstrahlung radiation. In this case several anodes may be used, for example a molybdenum anode with a characteristic emission line of 17.4 keV, a Zr with a characteristic emission line of 15.8 keV or a Nb anode with a characteristic emission line of 16.6 keV.

According to a preferred embodiment of the present invention irradiation system 3 is a scanning mechanism, which irradiates the tissue each time at a different location so as to obtain mapping of the prostate as further detailed hereinafter. Scanning irradiation systems are known in the art. For example one or more of the above-mentioned sources may be adapted for emitting the exciting radiation in a plurality of predetermined angles and/or a plurality of predetermined locations. The scanning of the tissue may also be performed manually by the operator by directing probe 1 to different directions and/or by positioning it at different locations.

Optionally, irradiation system 3 may be coupled to a monochromatizing element so as to provide a radiation with a substantially accurate (well defined) energy. Any suitable monochromatizing element may be used, including, but not limited to, a crystal monochromator or a plurality of different absorbing films each of which being characterized by a different absorption coefficient.

Apparatus 10 further comprises a radiation detector 6 located within probe 1 and capable of detecting emitted radiation 5. Detector 6 may have any shape compatible with the shape of probe 1, such as, but not limited to, a planar shape, a spherical shape, a cylindrical shape and the like. Detector 6 is preferably suitable for mapping emitted radiation 5, e.g., for the purpose of defining a boundary of a tumor 8 present in prostate 2. More specifically, detector 6 is preferably capable of detecting radiation from a plurality of predetermined angles so as to allow the mapping of the chemical element of interest. This may be achieved in more than one way. In one embodiment, detector 6 is a scanning detector, the scan of which is preferably synchronized with the scan of irradiation system 3. In another embodiment detector 6 is a position-sensitive detector which detects the emitted radiation as a function of its position. In an additional embodiment detector 6 is preferably an array of detectors (e.g., scanning detectors and position-sensitive detectors) being optimally arranged for detecting radiation as a function of position and/or angle.

Any known type of detector, which is suitable to detect the emitted radiation, may be used. For example, radiation detector 6 may be a high energy-resolution solid state detector such as, but not limited to, detectors based on Silicon (Si), Germanium (Ge), Silicon-Lithium-drifted (Si(Li)), Ge(Li), Mercury Iodide (HgI₂) or Cadmium-Zinc Telluride (CdZnTe), which can be cooled by a small thermoelectric device 54. Detector 6 may optionally be a high energy-resolution gaseous detector such as, but not limited to, a gas proportional detector or gas scintillation detector. It is to be understood that any other detector sensitive to X-rays in general and to a characteristic X-ray fluorescence emitted by chemical element 7 (shown as a zinc atom for the purpose of illustration only and without any intention of being limiting) is not excluded from the scope of the present invention. Detector 6 can optionally be a single element, a pixelized array or an array assembled of many individual elements. A solid state detector can optionally be a PIN diode, a surface barrier diode, a drift diode, a micro-strip detector, a drift chamber, a multi-pixel detector, a multi-strip detector and others. As shown in FIG. 2, apparatus 10 may also comprise electronic circuitry 52, to process signals from detector 6.

Thus, by detecting the radiation emitted from different locations of the prostate, apparatus 10 determines the level of the chemical element and thereby successfully maps the prostate. It is appreciated that such mapping is extremely important, for example, for the purpose of diagnosing prostate cancer. More specifically, apparatus 10 is capable of mapping the boundary of a prostate cancer according to a distribution of the chemical element in at least a region of the prostate, e.g., according to a distribution of different concentrations of the chemical element. In addition, the distribution of different concentrations of the chemical element may be used for staging the cancer so as to allow the physician to decide of an appropriate treatment. Alternatively or additionally, staging may be performed with a combination of different methods, optionally and preferably including analysis of needle-biopsy in vitro, and/or analysis of PSA as described below.

According to a preferred embodiment of the present invention apparatus 10 further comprises an X-ray optical system 19, located within probe 1, for the purpose of collimating and focusing the radiation emitted by irradiation system 3 and/or chemical element 7. As further detailed hereinunder, X-ray optical system 19 preferably prevents detector 6 from directly receiving any radiation emitted from irradiation system 3, and more preferably to receive only emitted radiation 5, which, as stated is emitted from chemical element 7. At least a portion of X-ray optical system 19 is preferably made of materials whose characteristic X-rays do not interfere with the determination of the tissue elements, in general, and Zn in particular.

Detector 6 is preferably in electrical communication (which can be either wireless communication or wired communication) with a signal recording, processing and displaying system 12 which maps the level of chemical element 7 in prostate 2 at a plurality of different points according to the mapping of detector 6. The mapping of system 12 may optionally be displayed on a display device (e.g., a monitor, a printer and the like) which is viewed by the operator for diagnostic purposes. For example, system 12 may be programmed so that zinc levels (or levels of any other chemical element) are graphically displayed on a two- or three-dimensional image of prostate 2, thereby to allow the operator to define the boundary of a cancerous region.

The electrical communication between system 12 and detector 6 is preferably controlled by electronic circuitry the size and shape of which is adapted to be compatible with the size and shape of probe 1. The electronic circuitry is designed and constructed for transmitting signals from detector 6 to system 12. The probe's head is preferably coated with a thin disposable polymer protection film 67, changed between examinations of different subjects.

The principles and operations of probe 1 can be better understood from FIGS. 2a-d which are schematic illustrations of the various components of probe 1, according to a preferred embodiment of the present invention.

FIG. 2a is a schematic illustration of probe 1. The beam containing radiation 4 is focused to a focal spot 55 having a preferred diameter of from about 0.5 to about 1 mm, behind a wall 58 (e.g., a rectal wall).

As stated, probe 1 comprises X-ray optical system 19 which preferably serves two purposes: (i) focusing and collimating the radiation emitted from irradiation system 3 (i.e., radiation 4) and (ii) collimating the radiation emitted from chemical element 7 (i.e., emitted radiation 5). According to a preferred embodiment of the present invention, system 19 may optionally comprise a focusing element 59 for performing the focusing functionality of system 19. Focusing element 59 may be, for example, a capillary optical device or an aperture having a suitable size. A preferred focal distance of focusing element 59 is from 70 mm to 100 mm. Focusing element 59 focuses beam 4 to spot 55.

In addition, system 19 preferably comprises a collimator 60 for performing the collimating functionality. The beam containing emitted radiation 5 (e.g., fluorescent radiation), emitted from a well-defined depth (focus point) is preferably collimated by collimator 60 into detector 6, which preferably has an annular geometry. Collimator 60 is preferably a multichannel device having a plurality of predetermined radiation paths 53, e.g., thin apertures, thin capillaries, X-ray optical elements and the like. A typical but non-limiting diameter of radiation paths is about 50-200 micrometer. Collimator 60 may have any geometrical shape, such as, but not limited to, a planar shape, a spherical shape or any other shape, as further detailed hereinbelow with reference to FIGS. 2c-d.

In any case the geometry of detector 6 preferably matches the geometry of collimator 60. For example, a spherical collimator is used with a spherical detector and a planar collimator is used with a planar detector.

Once collimated by collimator 60, emitted radiation 5 impinges on detector 6 which transmits the information via electronic circuitry 52 to system 12 (not shown in FIG. 2a). Probe 1 preferably comprises a thermoelectric cooler 54 being in contact with detector 6 for maintaining detector 6 at a sufficiently low temperature.

Broadly speaking, collimator 60 may be configured in more than one way. Hence, in one embodiment, collimator 60 directs radiation emitted from the chemical element in a single location to a plurality of locations on detector 6, in another embodiment, collimator 60 directs the radiation emitted from the chemical element in a plurality of locations to a plurality of locations on radiation detector 6, and in an additional embodiment, collimator 60 directs the radiation emitted from the chemical element in a plurality of locations to a plurality of detector-elements.

More specifically, as further demonstrated in the example section that follows, collimator 60 facilities the ability of detector 6 to discriminate between radiation emitted by the chemical element which is present in the prostate and radiation emitted by chemical elements which present in tissues surrounding prostate (e.g., rectal wall). For example, collimator 60 may be constructed so that radiation emitted by chemical elements present in tissues other than tissues of the prostate is filtered out. In particular, collimator 60 preferably collimates the size and/or divergence of the primary and the fluorescent beams, so that that the intersection of these beams defines a small volume within the prostate.

An additional realization of collimator 60 may be better understood from FIG. 2b, which is another schematic illustration of probe 1. In this embodiment, as further detailed herein below, detector 60 is capable of simultaneously detecting emitted radiation from a plurality of locations 55 in different depths inside the prostate.

Hence, according to a preferred embodiment of the present invention detector 60 comprises a plurality of predetermined radiation paths 53, each having a different size, so that radiation emitted by the chemical element present at different depths within the prostate is directed at different radiation detectors or different elements of a position sensitive detector. Specifically, each depth in the prostate is viewed by a circular array of detectors positioned at different radii. For example, let numeral 55′ represent an atom of the chemical element at a specific location. Atom 55′ emits emitted radiation 5′ which is collimated by path 53′ and detected by a predetermined location 6′ of detector 6. Thus, each depth corresponds to a predetermined region of detector 6, hence allows the identification of atom 55′ and its depth inside the prostate.

The attenuation of radiation from a specific location at large depth is preferably compensated by larger detector area at larger radius. It will be appreciated that the accuracy of the measurement is an increasing function of the number of locations from which radiation is detected. Thus, with the present configuration, both the accuracy of the measurement and the coverage of the prostate are substantially enhanced.

An additional advantage of collimator 60 is that the prostate may be mapped within a single measurement, thereby minimizing the need for manual or automatic scanning. In other words, as collimator 60 supports simultaneous measurement from a plurality of locations, the volume covered by probe 1 within a single measurement is substantially increased.

One ordinarily skilled in the art would appreciate that probe 1 may be manufactured from any material suitable for endoscopic procedure, such as, but not limited to, aluminum, plastics, polymers, carbon-fibers-based materials, Cu-free stainless steel. Generally, materials from which probe 1 is manufactured are preferably selected so that the characteristic lines of these materials do not conflict with the characteristic lines of the chemical element of interest. For example, if the chemical element is zinc, probe 1 is preferably manufactured from materials other than Cu or brass because of (i) the presence of Zn in brass; and (ii) the proximity of the Cu characteristic lines (8.04 and 8.904 keV) to that of Zn.

The external dimensions of the probe are preferably selected so as to optimize the active area of detector 6 while complying with the dimension of the cavity through which it is inserted (e.g., of the rectum). A preferred diameter of probe 1 for transrectal inspection is about 25 mm, which defines a sufficiently large detector area of about 100-200 mm², corresponding to a large detection solid angle. Large solid angles are needed for maximal reduction of the exposure time of inspection, by enhanced detection efficiency, keeping the radiation dose to the patient as low as possible.

Reference is now made to FIG. 2c, which is a schematic illustration of collimator 60 in a preferred embodiment in which collimator 60 is characterized by a planar geometry.

According to a preferred embodiment of the present invention collimator 60 comprises a planar plate 51 with collimating radiation paths 53 converging to spot 55 on one side and detector 6 on the other side.

FIG. 2d is a schematic illustration of collimator 60 in a preferred embodiment in which collimator 60 is characterized by a spherical geometry. In this embodiment, collimator 60 comprises a spherical plate 61 having a plurality of collimating apertures 65 converging to spot 55, a focusing element 59, which may be for example a capillary lens or an aperture of a suitable size, and a thin protective polymer film 67.

Preferred dimensions of collimator 60 include, but are not limited to, radii of forward and back spherical surfaces of the plate are about 6 and about 14 mm, radius of the spherical detector is about 15 mm.

It is estimated that the reduction of exposure time of probe 1 according to a preferred embodiment of the present invention, is about several hundred times in comparison with the single, small detector, presently used in standard X-ray fluorescence analysis systems. The focusing technique of the fluorescent radiation permits in-depth inspection, e.g., behind the rectal wall; the depth of the analyzed area is a function of the collimator distance from the wall. One would appreciate that in addition to the well-defined inspection geometry, the above configurations of collimator 60 strongly reduce the intensity of the scattered primary beam.

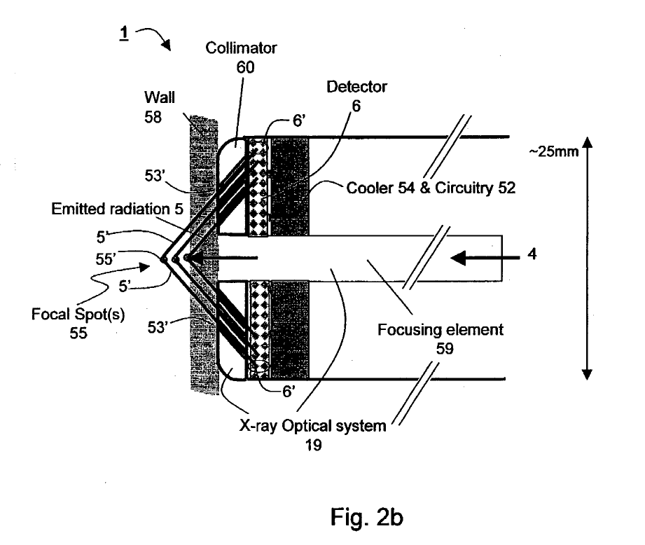

Reference is now made to FIG. 3, which illustrates a preferred use of apparatus 10, according to which probe 1 is introduced through the rectum in close proximity to the peripheral zone of prostate 2. A skilled artisan would appreciate that similarly probe 1, but with reduced dimensions, may be introduced through the urethra, in proximity to the central region of the prostate gland. Hence, a small area of prostate 2 is irradiated by the incident radiation 4 (not shown in FIG. 3) and the characteristic element X-rays 5 (not shown) emitted from element 7 are measured by the radiation detector. As the intensity of these X-rays is proportional to the concentration of the element in the prostate tissue, the level of the chemical element is measured. The operator then scans the prostate with probe 1 and obtains the distribution chemical element in the region under examination. This is of importance for staging of the prostate cancer. Alternatively, the use of an array of detectors or a position-sensitive detector, as further detailed hereinabove, eliminates the need for scanning and provides concentration mapping in a single measurement. It is estimated that with an optimized irradiation and detection setup, radiation exposure to the rectal wall of about 0.3 Roentgens will be required in order to detect Zn concentration in the prostate with a statistical precision of about 10%.

As stated, apparatus 10 may also be used for determining and mapping levels of chemical elements other than zinc, provided that such elements are detectable by XRF. Other chemical elements include, but are not limited to, elements normally present in the prostate gland tissue, e.g., iron (Fe), calcium (Ca) or bromine (Br), which may be detected separately or simultaneously with Zn for normalization purposes. As demonstrated in the Examples section that follows, the ratio of Zn/Fe in a cancerous prostate tissue is about 7 times lower than in normal prostate tissue. Thus, a normalization procedure, in which the level of one element is determined relatively to another element, referred to herein as a reference element, may provide information further distinguishing cancerous over normal tissues. Such normalization is known to be more accurate than a measurement of absolute concentration levels which may introduce inaccuracy due to dependence of the absolute levels on probe position (e.g., distance of the probe from the tissue), probe sensitivity and the like. A preferred normalization procedure for the purpose of qualitative determination of chemical element 7, comprises measuring the radiation emitted from element 7 in comparison to the radiation emitted from a reference element whose level is relatively constant. Alternatively the element concentration can be normalized to that of the Compton scattered part of the incident X-ray radiation.

Alternatively or additionally, apparatus 10 may be used for determining and mapping levels of chemical element introduced into the prostate for a specific medical procedure, e.g., palladium (Pd) in the form of Pd-porphyrin compounds and the like.

One such medical procedure is a photodynamic therapy (PDT), where one or more chemical elements (also known as photosensitizers) that bind to rapidly dividing cells are administered either directly to the prostate or systemically to the treated subject. The administrated photosensitizers have an inherent ability to absorb photons and transfer energy to oxygen which then converts to a cytotoxic or cytostatic species.

Referring now again to FIG. 1, according to a preferred embodiment of the present invention apparatus 10 may further comprise a device 14 for illumination of the prostate with light, which preferably has a wavelength suitable for exciting the administrated photosensitizers. Once excited, the photosensitizers induce a chemical reaction which results in a production of free radicals and/or other reactive products that destroy the abnormal tissue or cell with relatively small damage to the surrounding healthy tissue.

Thus, apparatus 10 has the advantage that it may be used for diagnostic purposes as well as for therapeutic purposes. The diagnosis and the therapy may be combined in a single treatment of the subject, where in a first stage the malignant tumor is detected and its boundary is defined and in a second stage the tumor is treated, e.g., using PDT. The diagnosis/therapy combination may be further facilitated by an injecting device 16 located within probe 1, for injection a drug or a contrast agent into the prostate. The contrast agent may be used, for example, for imaging purposes, when the use of apparatus 10 is combined with an imaging apparatus. The contrast agent may also be a chemical element which is known to bind to the cancerous region in the prostate. For example, if Pd is introduced to the prostate, the Pd may be used also for diagnosis and not only to be used for PDT.

Being equipped with detector(s) 6, apparatus 10 may optionally also be used for detecting radioactive substances (e.g., radioactive ¹²⁵I or Zn) introduced into the prostate for diagnostic purposes either systemically or by local administration into the prostate or proximal thereto. In such a mode of operation, the exciting radiation emanating from irradiation system 3 is typically turned off. This may optionally and preferably be done through a peripheral device or through an ON/OFF switch included within probe 1. The measurement of radioactive substances may be useful for staging the disease, as for example it is known that changes in the ¹²⁵I concentration levels in the prostate may indicate a cancerous pathological condition of the prostate.

It is appreciated, that in some cases, the diagnosis of the detected tumor may have some degree of inconclusiveness, and that in such cases the real-time diagnosis should be supplemented by biopsy. In other cases, apparatus 10 is used for the purpose of locating a region of the prostate (e.g., when probe 1 is used as a radioactive detector) from which a biopsy is to be taken. In any case, according to a preferred embodiment of the present invention, apparatus 10 preferably comprises a biopsy device 18 for performing biopsy from a specific region of the prostate.

According to a preferred embodiment of the present invention, probe 1 is combined with or comprises an additional mapping device 17, such as, but not limited to, an ultrasonic device, a magnetic-resonance-imaging device. In this embodiment, apparatus 10 is capable of mapping the prostate by XRF and also preferably by an additional method (e.g., ultrasonic waves). The advantage of such a double mapping procedure lies in the enhanced accuracy of determining the tumor location, so that the number of biopsies (if any is required) is minimized. In contrast, presently known TRUS procedures have low reliability and repeated biopsies are needed, with the risk of infections and extra costs.

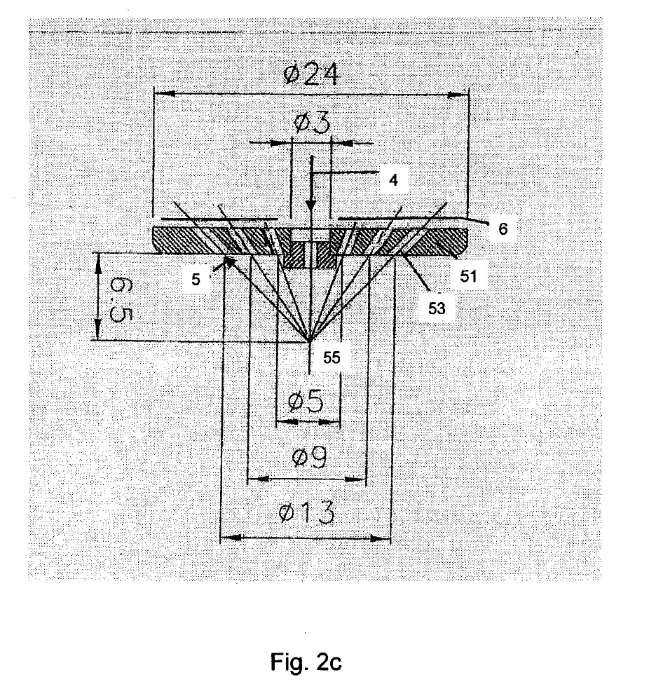

According to another aspect of the present invention there is provided a method of non-invasive in vivo detection of a chemical element in the prostate of a subject. The method comprises the following method stages, which illustrated in the flowchart of FIG. 4a and can be performed, for example using apparatus 10.

Referring to FIG. 4a, in a first stage, designated by Block 31, a probe (e.g., probe 1) is endoscopically inserted into the subject, e.g. through the rectum or the urethra. In a second stage, designated by Block 34, the probe is used for irradiating the prostate by exciting radiation so as to excite the chemical element to emit fluorescent X-ray radiation. Optionally, the chemical element may have been introduced to the prostate prior to the process of irradiation, as previously described. The exciting radiation may be generated by known means such as, but not limited to, irradiation system 3. In a third stage, designated by Block 36, the probe is further used for detecting and mapping the emitted radiation, for example using detector 6, or an array of detectors, as further detailed hereinabove.

The detection and mapping of the emitted radiation is preferably performed by first determining the intensity of emitted radiation; and (ii) calculating the level of each of the elements in the prostate from which radiation was emitted based on the measured intensity of the radiation. As stated, the level of an element may be determined either by absolute radiation levels, or by relative levels, using the above-mentioned normalization technique.

In a fourth stage, designated by Block 38, the level of the chemical element in the prostate of the subject is mapped at a plurality of different points in the prostate according to the mapping of the emitted radiation.

Additional optional stages of the present method include, but are not limited to, using the probe for (i) performing a biopsy procedure, preferably a needle biopsy procedure (Block 39); (ii) injecting a drug or a contrast agent into the prostate (Block 40); and (iii) illuminating the prostate with light (Block 44), e.g., for the purpose of photodynamic therapy, as detailed hereinabove. Optionally and preferably, the method may further comprise a stage (Block 45) in which the prostate is mapped by a method other than XRF, as further detailed hereinabove. It should be noted that any of the above optional stages may optionally be performed in any order within the method.

An additional optional but preferred method and system of the present invention relates to the correlation between the levels of the chemical element in the prostate and prostate-specific-antigen (PSA) analysis. According to this aspect of the present invention, at least two parameters are determined, including a first parameter that may optionally represent the level or concentration of a chemical element, and a second parameter that is preferably indicative of PSA activity in the blood serum of the subject. The two parameters may optionally be determined by appropriate apparatuses or devices, e.g., apparatus 10 for determining the level of a chemical element and an additional apparatus for determining the second parameter, which is commonly known to in the art. Alternatively, the determination of the level of the chemical element may also optionally be done by needle-biopsy, i.e., in vitro.

Alternatively, as shown with regard to FIG. 4b, the determination of the level of the chemical element may also optionally be done by needle-biopsy, i.e., in vitro. According to further preferred embodiments of the present invention, the analysis of tissue by needle-biopsy may optionally be combined with the previously described in vivo probe analysis. For example, an area of tissue in the prostate may be determined to be possibly cancerous, or to otherwise require further diagnosis, because of the mapping process performed with the apparatus according to the present invention. A needle-biopsy may then optionally be obtained from the area requiring further diagnosis, and may then also optionally and preferably be analyzed according to the method of the present invention in vitro. For this purpose, the apparatus of the present invention may optionally and preferably be adapted to measure the level of the chemical element in the tissue obtained through the needle biopsy.

Hence, according to a preferred embodiment of the present invention, in a first stage, designated by Block 46, a biopsy is preferably performed to remove a portion of the prostate. In a second stage, designated by Block 47, the level of the chemical element in the tissue obtained by the biopsy procedure is measured. Optionally and preferably, the method comprises an additional stage, designated by Block 48, in which the level of the chemical element is correlated with the second parameter.

A preferred method for the determination of the level of the chemical element (either in vivo or in vitro) is by XRF, as further described hereinabove. Alternatively, or additionally, the determination of the level of chemical element may optionally be performed through activation analysis, atomic absorption or particle-induced X-ray emission, or a combination thereof.

Once the parameters are determined, the prostate cancer is preferably diagnosed by a set of rules. For example, it has been found by the inventors of the present invention that a prostate cancer may be accurately diagnosed, if the first parameter (the level of the chemical element) is below one predetermined threshold and the second parameter (the PSA indicative parameter) is above another predetermined threshold. A skilled artisan would appreciate that whether or not a certain parameter is above or below its respective threshold depends on the parameter and could easily be determined according to medical and/or biological functions that are well known in the art. Hence, it is not intended to limit the scope of the present invention to any specific rules, and other relations between the parameters and the thresholds may be used. For example, if the chemical element is introduced so that its concentration is enhanced in cancerous regions, the cancer may be accurately diagnosed if the level of the chemical element is above a certain threshold.

According to a preferred embodiment of the present invention the second parameter may be serum PSA level, PSA density, PSA velocity, a level of age specific PSA or percentage of free PSA. In case of serum PSA level it has been found that a threshold of about 4 ng PSA/I serum and in case Zn is the chemical element to be detected, the preferred threshold can be 80 μg zinc/g prostate tissue. It should be understood, however, that other thresholds (for all the parameters) are not excluded from the scope of the present invention.

One of ordinary skill in the art would appreciate the advantage of the use of more than one discriminator for cancer diagnosis. As demonstrated in the Examples section that follows, the combination of two or more parameters provides a clear improvement on the diagnostic value of each of them separately. For example, by using two discriminating parameters it has been found by the inventors of the present invention that the percentage of false-positive diagnoses is reduced from about 45% to about 18% (data shown in the Examples section that follows).

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### In Vitro Measurements of X-Ray Spectrum of Prostate Phantom

The determination of the optimal energy of the incident radiation and a feasibility of the in vivo Zn determination according to the invention was demonstrated in the laboratory using a prostate phantom in the form of a polyethylene vial filled with aqueous Zn solution.

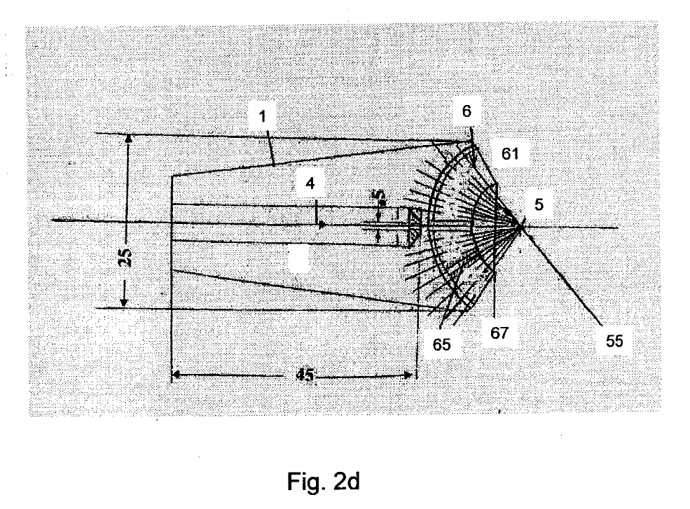

The experimental arrangement is shown in FIG. 5. The irradiation system was a filtered X-ray beam 20 from a tungsten anode X-ray tube 22. The tube 22 was operated at 36 kV and the filter 24 was a combination of Cu/Mo foils. The diameter of the beam on the sample 26 was about 10 mm. A ferrous collimator 28 was included in the beam's path. The irradiated samples consisted of 30 cc polyethylene vials, 34 mm in diameter and 1 mm thick wall, containing aqueous solutions of Zn. The Zn characteristic X-rays were emitted from the sample and detected by Si(Li) detector 30 (5 mm² in area) cooled by a liquid nitrogen (LN₂) arrangement 32.

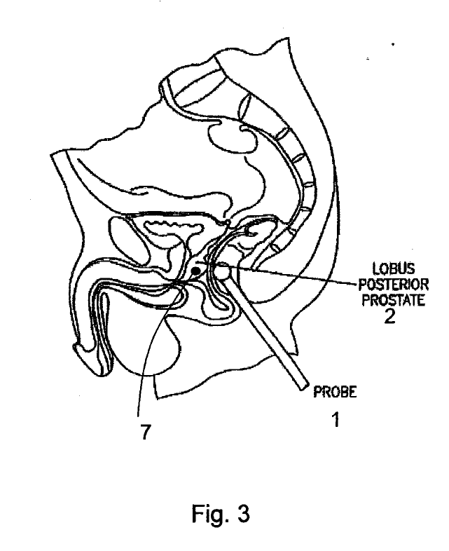

FIG. 6 is a graph showing a spectrum obtained from irradiation of a vial containing 1000 μg/g of Zn aqueous solution. The vertical axis of the graph represents the logarithm of the number of counts and the horizontal axis represents the energy. As can be observed the Zn peak is very well defined and is positioned on a flat background. The small peak on the left side is due to Cu most probably present in the detector structure or housing. From this preliminary study it can be concluded that the optimal source X-ray energy for detection of Zn is in the range of 13-18 keV.

The complexity of the detected X-ray spectrum was tested in vitro using the XRF method on prostate tissue-samples and indicated that Zn in prostate can be accurately measured without interference from other elements. The samples were exposed to X-rays emitted from an X-ray tube with a Mo anode. The tube operated at 27 keV and the characteristic Mo X-ray line of 17.44 keV was filtered out using a crystal monochromator. The characteristic radiation emitted from the sample was measured using a Si(Li) detector having energy resolution of 160 eV at 6.4 keV.

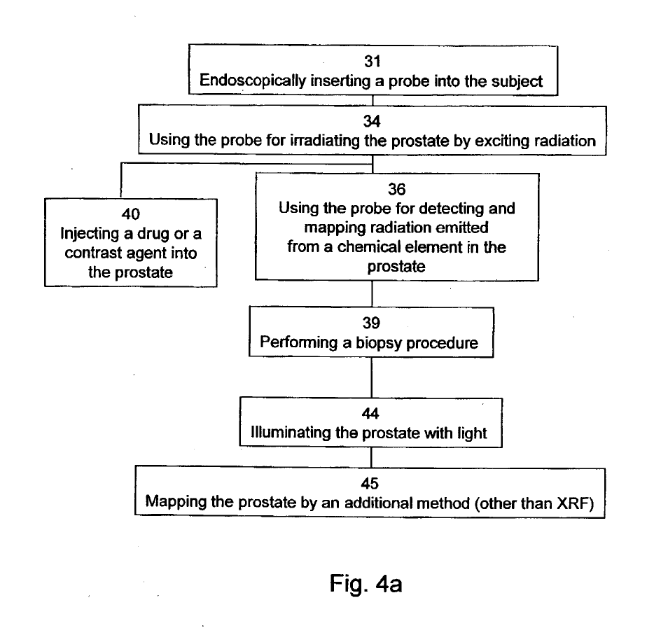

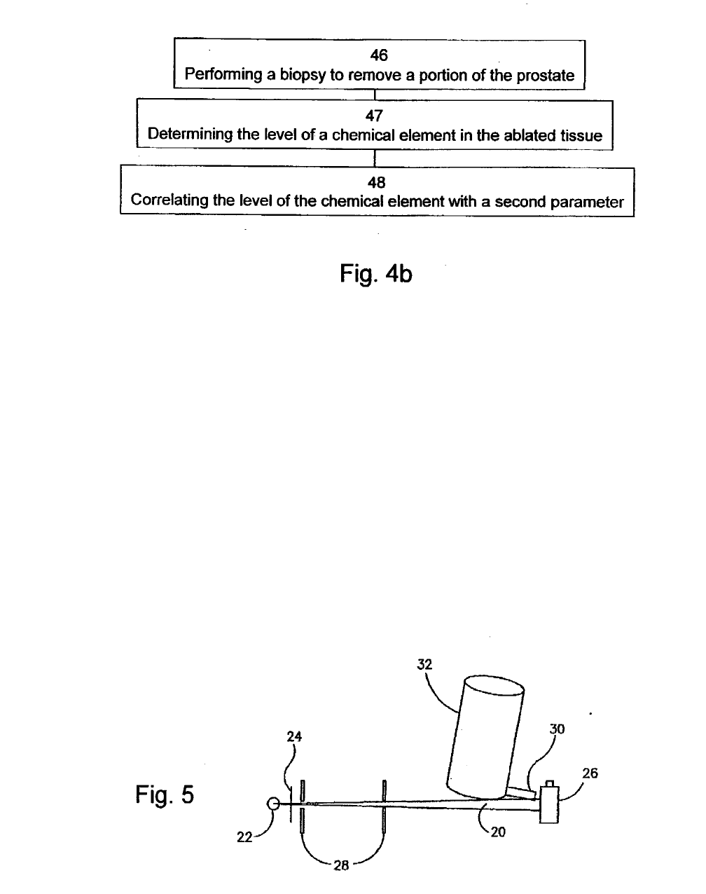

FIG. 7 and FIG. 8 show the XRF spectra obtained from prostate specimens embedded in paraffin, prepared for histological examination. Both spectra are shown as the logarithm of the number of counts as a function of the energy. As can be observed from the spectra, the Zn concentration in the cancerous tissue is much smaller than in the prostate with adenoma. Other elements such as Ca and Fe are also measurable and can be used for normalization purposes. For example, the ratio of Zn/Fe in the tissue was about 7 times lower in the case of prostate cancer.

### Example 2

### XRF and PSA Measurements in Fresh Prostate Tissues

The purpose of the present Example is to examine the possibility of using prostate Zn concentration as a base for an in-vivo diagnostic procedure. An XRF facility optimized for Zn measurement in fresh prostate tissues was constructed. For the purpose of exploring the diagnostic potential of using Zn levels determined either from biopsies or non-invasive in-vivo methods in combination with other indicator, such as PSA, the fresh prostate tissues were subjected to histological examination as well as to the XRF facility.

Prostate samples from a total of 28 patients were analyzed in this study. Clinical records included age, serum PSA, previous medical therapy, surgical procedure and histology. Prostate tissue samples were obtained from patients undergoing surgical procedures: suprapubic prostatectomy (SPP) in 21 patients for BPH; radical prostatectomy (RRP) in 6 patients for prostate cancer (CAP) and radical cystoprostatectomy (RC) in 1 patient for bladder cancer. Due to the different surgical procedures employed, the tissues under examination actually originate from different locations within the prostate: SPP yielded tissues from the posterior part of the transitional zone, whereas those obtained via RRP and RC originate in the posterior part of the peripheral zone. Two tissue samples were obtained for each prostate. Immediately following the operation, each sample was dissected into two parts. One part was used for histological examination and kept in formaldehyde. The second part was frozen and used for determination of zinc content by XRF.

Histological Examination

Tissue Preparation

The tissue parts kept for histological examination were prepared for conventional optical microscopy using standard procedures. Each sample was fixed in 4% buffered formalin; embedded in paraffin wax; cut into 4 micrometers thick sections and stained with hematoxylin and eosin.

Description of Analysis

The following histological features were recorded: histological diagnosis, Gleason score [Gleason D: Classification of prostate carcinoma. Cancer Chemotherapy Report 1966;50:125] (in case of prostatic carcinoma) and percentage of glandular tissue; the latter was estimated as a percentage of the total sample surface. On the basis of pathological examination, the samples were categorized in three groups: BPH, CAP and focal cancer in addition to BPH (BPH/CAP).

Determination of Zn by XRF

Tissue Preparation

Tissue samples, 1 cm in diameter and 1-2 mm thick were stored prior to measurement at −70° C. During the measurement the sample was either maintained at −8° C. (in a cooling chamber with a thin 2.5 μm thick Mylar window) for measurement times exceeding 15 minutes, or at room temperature (between two 2.5 μm thick Mylar foils) for short measurements of 5 minutes. Besides freezing, no tissue preparation is required or recommended for this measurement. Repeating the measurement, after storage time of one month, yielded the same results.

Apparatus for Measurement of Zn by XRF

The samples were irradiated with an X-ray tube, having a W or Mo anode. The tube operated at 30 kV and at currents of 30 or 10 mA. The primary beam was filtered using a 150 or 50 μm thick Mo foil. The beam was collimated using a 1-3 mm aperture such that the diameter of the beam impinging on the sample was not more than about 5-6 mm. Several measurements were taken across each sample. The radiation from the sample was measured using an Amptek XR-100CR Pelltier-cooled Si PIN detector positioned at angles of ˜120° or ˜90° relative to the incident beam direction. The energy resolution of the detector was 240 eV at 5.9 keV. The system was calibrated using standards consisting of tissue-equivalent solutions with known Zn concentrations.

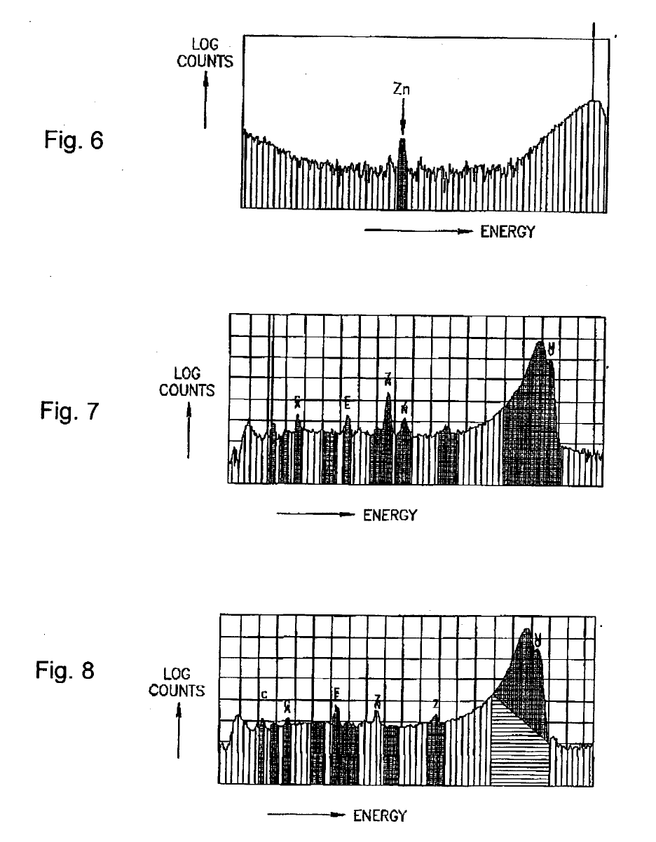

FIGS. 9a-b exemplify XRF spectra (counts as a function of the energy) of Zn content in prostate samples. FIG. 9a shows an XRF spectrum from a sample diagnosed as CAP (patient # 14), and FIG. 9b shows a spectrum from a sample diagnosed as BPH (patient #19), with designation of the peaks at energies corresponding to Zn and Sc. As shown in FIGS. 9a-b, the peak of the sample diagnosed as BPH is substantially higher than the peak of the sample diagnosed as CAP.

Results

The histological examination consisted of diagnosis and Gleason score. Table 1 shows the results of the diagnosis and the Gleason score.

TABLE 1

| Patient No. | Age [y] | Surgery type | Diagnosis | Gleason Score |
|---|---|---|---|---|
| 1 | 73 | SPP | CAP | 5(2 + 2) |
| 2 | 79 | SPP | BPH | |
| 3 | 69 | SPP | BPH | |
| 4 | 65 | RRP | CAP | 6(3 + 3) |
| 5 | 69 | SPP | BPH | |
| 6 | 64 | RRP | CAP | 5(3 + 2) |
| 7 | 66 | RC | BPH/CAP | |
| 8 | 91 | SPP | BPH | |
| 9 | 45 | SPP | BPH | |
| 10 | 90 | SPP | BPH | |
| 11 | 54 | SPP | BPH | |
| 12 | 77 | SPP | BPH | |
| 13 | 79 | SPP | BPH | |
| 14 | 68 | RRP | CAP | 8(5 + 3) |
| 15 | 76 | SPP | BPH | |
| 16 | 68 | SPP | BPH | |
| 17 | 78 | SPP | BPH/CAP | 4(2 + 2) |
| 18 | 80 | SPP | BPH/CAP | 4(2 + 2) |
| 19 | 72 | SPP | BPH | |
| 20 | 90 | SPP | BPH | |
| 21 | 70 | SPP | BPH | |
| 22 | 70 | SPP | BPH | |
| 23 | 78 | SPP | BPH | |
| 24 | 67 | RRP | CAP | 5(3 + 2) |
| 25 | 81 | SPP | BPH | |
| 26 | 75 | SPP | BPH/CAP | 4(2 + 2) |
| 27 | 66 | RRP | CAP | 6(3 + 3) |
| 28 | 68 | RRP | CAP | 6(3 + 3) |

The histological analysis revealed 7 cases of CAP and 17 cases of BPH. In four patients (#7, #17, #18 and #26) there was histological evidence of focal cancer in addition to BPH.

Table 2 shows the PSA values along with the Zn concentration in the sample, in units of μg/g wet weight, as the PSA as determined by the XRF method. As the measured Zn concentration varied significantly between the two samples, the results here show the lower Zn value between the two. The justification for this choice is the possibility that a local minimum in Zn concentration may indicate a localized cancer region.

TABLE 2

| Patient No. | Diagnosis | PSA (ng/l) | Zn (μg/g) |
|---|---|---|---|
| 1 | CAP | 5.4 | 36 |
| 2 | BPH | 1.5 | 120 |
| 3 | BPH | 7.6 | 58 |
| 4 | CAP | 6.1 | 15 |
| 5 | BPH | 9.5 | 56 |
| 6 | CAP | 5.8 | 15 |
| 7 | BPH/CAP | 5.9 | 46 |
| 8 | BPH | 18 | 111 |
| 9 | BPH | 1.2 | 35 |
| 10 | BPH | 0.7 | 7 |
| 11 | BPH | 9.8 | 85 |
| 12 | BPH | 3.3 | 92 |
| 13 | BPH | 2.2 | 72 |
| 14 | CAP | 5.8 | 46 |
| 15 | BPH | 1.5 | 69 |
| 16 | BPH | 9.7 | 72 |
| 17 | BPH/CAP | 3.1 | 143 |
| 18 | BPH/CAP | 1.4 | 54 |
| 19 | BPH | 7.3 | 173 |
| 20 | BPH | 3.5 | 204 |
| 21 | BPH | 2.2 | 157 |
| 22 | BPH | 11.5 | 139 |
| 23 | BPH | 8.2 | 329 |
| 24 | CAP | 4.9 | 49 |
| 25 | BPH | 2.8 | 106 |
| 26 | BPH/CAP | 3.9 | 351 |
| 27 | CAP | 8.3 | 71 |
| 28 | CAP | 6.5 | 44 |

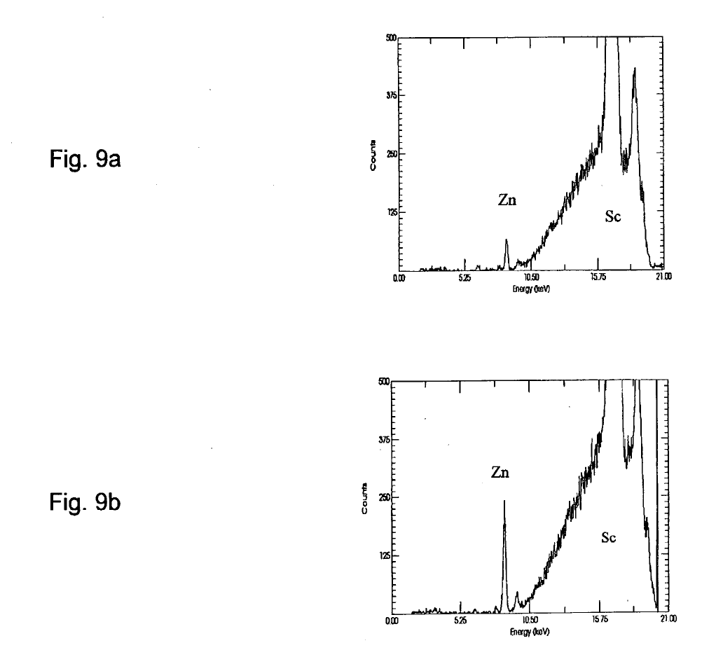

FIG. 10 shows a graphical representation of the above Zn concentrations in the prostate sample for BPH, CAP and CAP/BPH, where each subject is represented as a point on the graph, depending on the diagnosis (horizontal axis) and the detected Zn concentration.

As can be observed, the scatter of results in each category is large and a degree of overlap between BPH and CAP exists. The mean Zn value and standard deviation of the mean for CAP, BPH and CAP/BPH are 40±8, 110±17 and 148±71 μg/g, respectively. The difference between CAP and BPH groups has a significance level p=0.025. If a lognormal distribution of Zn values is assumed the p-value is further reduced to 0.018. There is no significant difference between BPH and BPH/CAP groups.

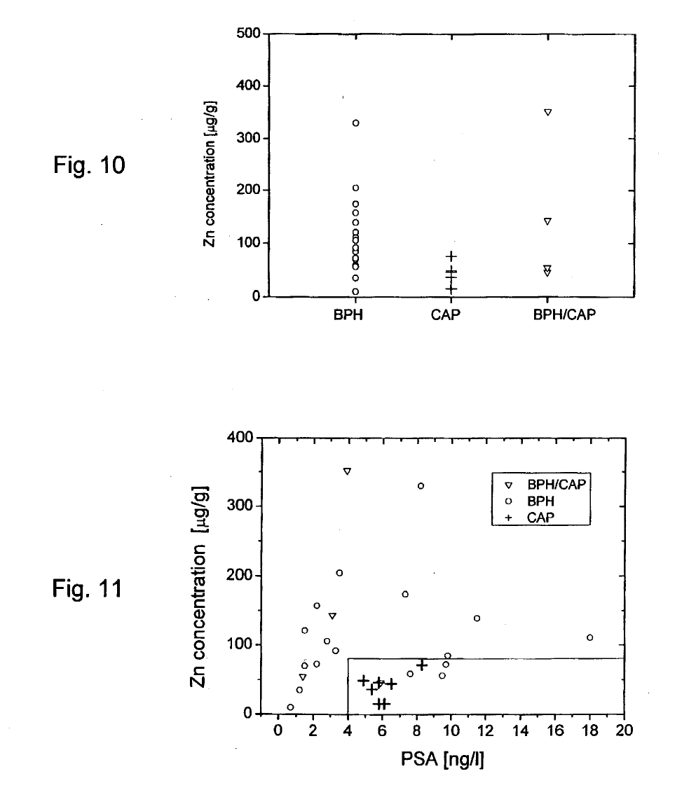

FIG. 11 is a graph demonstrating the correlation between the lower Zn content and the PSA value. The horizontal axis of FIG. 11 represents PSA values (ng/l) and the vertical axis represents Zn concentration (jg/g). Here an interesting pattern should be noted. One can divide the Zn vs. PSA behavior in BPH into two regions. Between PSA values of 0-4 ng/l, the Zn content rises sharply with PSA and peaks at around 4 ng/l. Above 4 ng/l, Zn values seem to have large scatter, but it would appear that there is a gradual decrease of Zn with increase in PSA. The relation in CAP cases does not show this pattern. All the CAP cases seem to be concentrated in 5-9 ng/l region and each case the Zn value is below 80 μg/g. Except in one case, the BPH/CAP cases do not differ in behavior from cases of BPH. Above PSA value of 4 ng/l the difference in Zn values for BPH and CAP groups had a much higher significance of p=0.0036 using a lognormal distribution.

The Zn-PSA relationship is interesting because it enables more efficient discrimination between benign and malignant prostate. For example by using only the PSA values and setting its lower limit at 4 ng/l, all of the cancer cases are detected but the detection would also include also 47% of the BPH cases (47% false positive). However, if in addition to the PSA threshold, an upper limit threshold for Zn concentration is set at 80 μg/g, the rate of false positive cases is reduced to 18%. Thus by setting a region (as shown by the smaller rectangle inside the graph of FIG. 11) contained between boundaries Zn<80 μg/g and PSA>4 ng/l an area which may have a high value in accurate diagnosis of CAP is defined.

Discussion

The different surgical procedures applied in this example resulted in tissue samples originating from different prostatic zones. In most cases the surgical procedure was SPP, for which the analyzed tissue originated from the posterior part of the transitional zone. Upon histological examination, only one of the 21 SPP-operated cases revealed diffuse CAP (+three focal CAP). In contrast, 6 out of 7 diagnosed CAP cases resulted from analysis of the peripheral zone accessible through the TRUS biopsy and RRP procedures. Zn “mapping” of the peripheral zone with an in-vivo probe could therefore be more useful when evaluating the prostate gland for the presence of malignancy than the analysis of the transitional zone.

As expected, the average Zn concentration in cancer is lower than in BPH, however there were large variations of Zn concentration within the prostate tissue indicating that multi-sampling is necessary.

No correlation between Zn concentration in CAP and the Gleason score, which relates to tumor grading, or to the local extent of the tumor, was observed.

The results show considerable overlap of the BPH and CAP Zn values. There are a substantial number of BPH cases with low (<80 μg/g) Zn concentration. Thus, a low Zn concentration value is not necessarily an indication of CAP and, if used as a sole diagnostic indicator, will cause a substantial number of false positive diagnoses (41% in the data).

The most interesting finding of the present example is the relationship between Zn concentration and PSA levels (FIG. 11). The combination of these parameters is a clear improvement on the diagnostic value of each of them separately. In the presented data, if an upper level boundary of 80 μg/g is applied to the Zn concentration all CAP cases and two BPH/CAP cases are detected with 41% false-positive diagnoses. On the other hand, by using PSA only with a lower boundary of 4 ng/l, all CAP cases and one BPH/CAP are detected, with 47% false-positive diagnoses. Combination of the two indicators maintains the CAP detection level but reduces the false-positive diagnoses level to ˜18%.

On the graph of PSA-Zn relationship (FIG. 11) the three cases of CAP/BPH are indistinguishable from BPH. This finding could contradict the suggestion made by Habib et al. and other investigators that a decrease in Zn is an early step in malignancy.

In addition to using serum PSA levels, as shown above, it is also possible to use other parameters of PSA activity, such as PSA density, PSA velocity, age specific PSA and percent of free PSA.

The above findings support the idea that Zn could aid CAP diagnosis and substantially reduce false-positive diagnoses. Thus in-vitro or in-vivo measurements of prostatic Zn are of importance. Other methods of Zn detection may be used, in addition to X-ray fluorescence (XRF), such as activation analysis, atomic absorption, and particle-induced X-ray emission (PIXE).

### Example 3

### In-Depth Topographic Zn Determination of Prostate Phantom

The in vivo measurement of Zn in the prostate through the rectum, according to preferred embodiments of the present invention, involves a non-trivial assessment of Zn concentration within the prostate while traversing a few millimeter thick rectal wall that also contains Zn, but at lower levels. The concentration of Zn in non-prostatic tissue surrounding the prostate is known to be about 12 times smaller than that in the dorsal lobe of a non-malignant prostate. The attenuation coefficient for 8.6 keV in tissue (8.3 cm⁻¹ and absorption length of 1.2 mm) results in a significant attenuation of the exiting fluorescence radiation through a 3 mm thick rectal wall. It is estimated that the rectal wall attenuates the radiation by a factor of 12. At such high attenuation the contribution of Zn from tissues other than prostate, such as the rectal wall, can become significant and mask the signal from the prostate Zn.

One way to overcome this problem is to limit by collimation both the size and divergence of the primary and the fluorescent beams, such that their intersection defines a small volume within the prostate, behind the rectal wall, and the XRF radiation is detected only from this volume. Such technique is known in the art, e.g. from determination of the content of micro samples by means of XRF topography and from observation of defects inside single crystals by means of X-ray diffraction topography. The main goal of the present example is to demonstrate the use of XRF topography in view of the determination of Zn in prostate in-vivo.

Methods

The XRF Facility and Phantoms

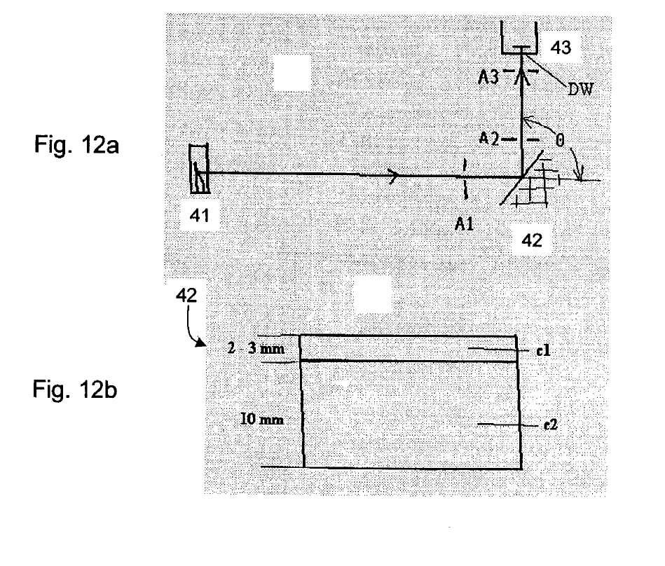

FIG. 12a shows the experimental system, which included a constant potential X-ray tube 41, a phantom 42 and a detector 43. Three apertures, designated A1, A2 and A3, were positioned in the path of the X-ray radiation. Also shown in FIG. 12a are the detector entrance window, designated DW and the angle between the primary and fluorescent beams, designated in FIG. 12a by Θ.

The X-ray tube (PW2275/20) has Mo anode, and a focal size of 1×10 mm. The tube was operated at 30 keV and 30 mA. A 50 μm thick Mo foil was used to filter out most of the bremstrahlung radiation such that the dominant incident radiation was the 17.4 keV Mo-K_{α} line. The primary and fluorescent beams were collimated to diameters of 0.5-2 mm, by apertures A1, A2, and A3; the distances between the different elements were as follows: 41−A1=400 mm, A1−42=20 mm, 42−A2=40 mm and A2−A3=40 mm. The diameter of apertures A1, A2 and A3 were 0.5, 0.8 and 4 mm, respectively. In these conditions, the divergence of the primary beam was about 10 mrad (horizontal) and 2 mrad (vertical) and the divergence of the fluorescent beam was about 20 mrad in both directions.

The fluorescent radiation from the samples was measured with an Amptek XR-100CR Peltier-cooled Si PIN detector (designated 43 in FIG. 12a), placed at angles of Θ=90′ or Θ=150°. In typical fluorescence spectra obtained in-vitro from a human prostate tissue (see FIGS. 9a-b in Example 2) one can observe Zn lines (at energies 8.64 and 9.57 keV) together with scattered radiation at higher energies. In addition, there are weak lines of Fe and Ni. These lines most probably originate from fluorescence of the stainless steel entrance window frame of the detector, induced by the Compton radiation scattered from the sample.

FIG. 12b shows phantom 42 which comprises two flat containers, 15 mm in diameter, filled with tissue equivalent solution containing known Zn concentrations. The first container (2 mm thick) was filled with low Zn concentration (designated c₁ in FIG. 12b) solution, modeling the rectal wall tissue, and the second container (10 mm thick), with high Zn concentration (designated c₂) solution, modeling a normal prostate tissue. The composition of the tissue equivalent solution was (64% H₂O, 28% glycerol, 7% urea, 0.3% NaCl and 0.7% K₂S₂O₈ weight fraction) to which Zn chloride was added. In order to speed up the time of the measurements, high Zn content of 8000 μg/g in the prostate compartment was employed here. The windows of the containers were made of 2.5 μm thick Mylar foil, in which the absorption of Zn fluorescent radiation is negligible.

Configuration of the XRF Topography System

The principle of XRF-topography is based on the registration of fluorescent radiation originating only from a well-defined small volume within an object.

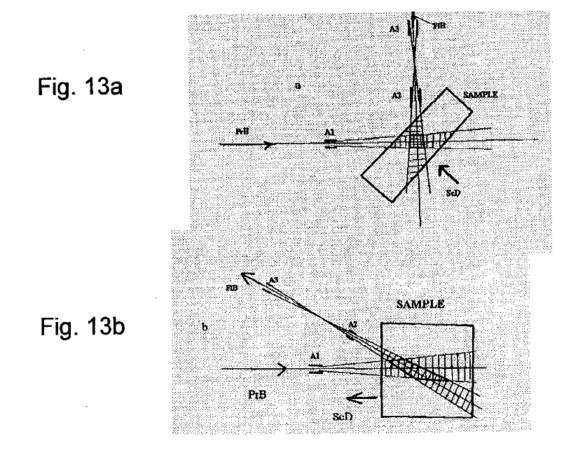

FIGS. 13a-b show the scheme of the beams crossing inside the object for scattering angles of Θ=90° (FIG. 13a) and Θ=150′ (FIG. 13b). In FIGS. 13a-b, the primary beam is designated PrB, the fluorescent beam is designated FIB, and the scanning direction is designated ScD.

The shape and location in space of the inspected part within the object (focal volume) are determined by the intersection of two cones. A first cone is of the incident primary beam formed by aperture A1 (marked by vertical shading in FIGS. 13a-b). A second cone is of fluorescent radiation, which is visible by the detector through apertures A2 and A3 (marked by lines perpendicular to the fluorescent beam FIG. 13a).

The configuration shown in FIG. 13a (Θ=90°) defines a smaller focal volume and provides a better spatial resolution in the depth direction. The configuration shown in FIG. 13b (Θ=150°) is closer to the geometry that has to be used in the in-vivo probe. In the latter the shape of the inspected volume is more extended and the spatial resolution along the depth direction is worse. By moving the sample or the source-and-detector system, one can scan the sample in the depth direction and get the fluorescence signal as function of depth. In this experiment the phantom was moved and the source-and-detector system was kept stationary. At the 90° configuration the scanning direction was along a bisector of the angle between the primary and fluorescent beams. At 150° the movement of the sample was along the direction of the incident beam.

Determination of the Response Function of the System

In order to determine the intrinsic spatial resolution of the system in the scanning direction, the phantom shown in FIG. 12b was replaced by a 0.3 mm thick Cu foil. The characteristic energy of the Cu line (8.04 keV) is close to that of Zn and the effective thickness of the fluorescent layer is about 20 μm. A scan obtained with this foil essentially provides the response of the system to a very thin layer of an element of interest, i.e., the point spread function of the system, and can be utilized for the analysis of data obtained from more complex phantoms.

Phantom Studies

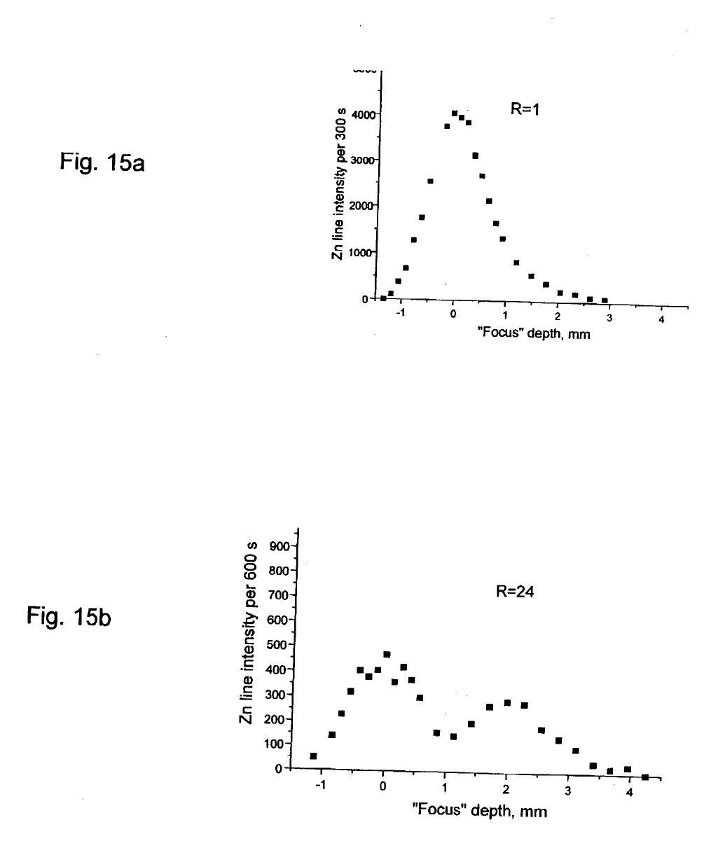

The phantom studies consisted of performing in-depth XRF scans of the layered phantom described above, that simulates the rectal wall (2 mm thick) and the prostate (10 mm thick). The two compartments were filled with solutions (aqueous or tissue equivalent) of different Zn content. The Zn concentration in the prostate compartment was always kept constant at 8000 μg/g. A ratio R defined as c₂/c₁ between Zn concentrations in the “prostate” and the “rectal wall” compartments was varied from 1 (uniform phantom) to 3, 6, 12 and 24 for FIGS. 15a to 15e, respectively. These scans were performed at the 90° configuration.

In order to simulate a more realistic in-vivo Zn measurement situation, one scan was performed with the 150° configuration using a phantom containing tissue equivalent solution with Zn concentrations of 250 μg/g and 1000 μg/g in the rectal wall and the prostate compartments, respectively. In this experiment the phantom was positioned perpendicularly to the incident beam and the scan was performed along the incident beam direction.

Results

Response Function

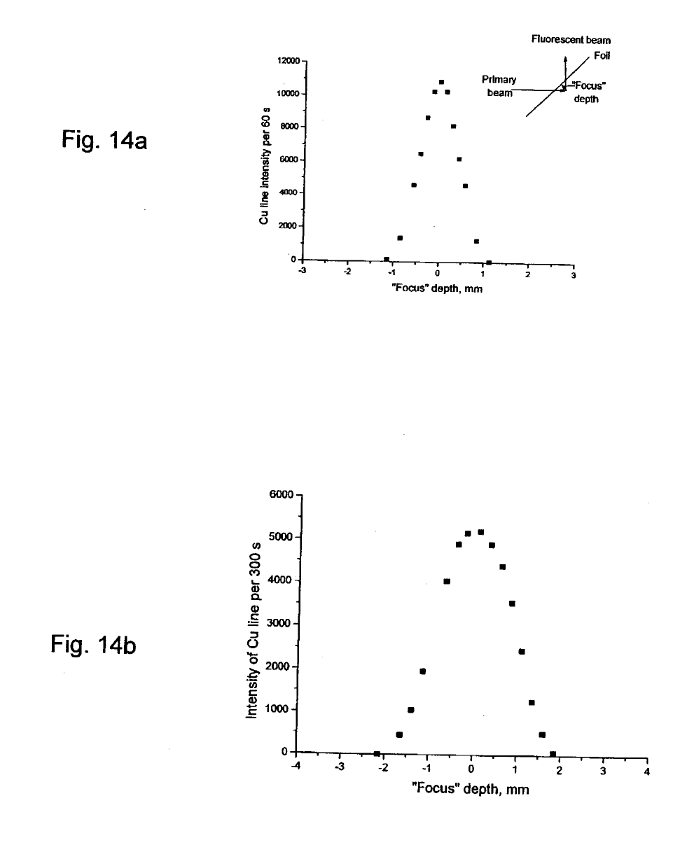

The experimental result of scanning the Cu foil with the 90° geometry, is shown in FIG. 14a, where the horizontal axis represents the depth in mm and the vertical axis represents the intensity of the Cu line. When the foil is centered at the crossing point of both beams (“focus” position), the fluorescent radiation intensity is maximal. The resulting scanning curve is symmetric, with full width at half maximum (FWHM) of about 1 mm, representing the intrinsic resolution of the method in the depth direction under the present conditions. A response function for the 150° configuration is showed in FIG. 14b. The diameters of the apertures A1 and A2 were 2 mm and 1 mm, respectively. The response function for this geometry is broader and has FWHM of 2 mm.

Phantom Scans

FIGS. 15a-e show the results of phantom scans carried out at the 90° geometry, for the above values of the Zn concentration ratio R, where the horizontal axis represents the depth in mm and the vertical axis represents the intensity of the Zn line. The results were obtained by measuring the intensity of the 8.6 keV K₊ line from different depths within the phantom.

FIG. 15a shows a scan through a uniform phantom (R=1). As the sensitive focal volume (created by the intersection of the incident and recorded fluorescent beams), enters into the phantom, the number of counts increases; after reaching a maximum it starts to decrease due to the self absorption of the 8.6 keV Zn fluorescent radiation at larger depths, forming a tail. One can observe that the intensity decreases rapidly with depth. The scanning curve shown on FIG. 15a is characteristics of a homogeneous object. Such situation may occur in case of cancerous prostate in which the Zn content may decrease to the levels of non-prostatic tissue in its surrounding, such as the rectal wall.

FIGS. 15b-e show the scanning results of the two-compartment phantoms, with R=24, 12, 6 and 3, respectively. In all the experiments shown in FIGS. 15b-e, the following numerical values were used: c₁=c₂/R, c₂=8000, 2 mm thickness for the “rectal wall” compartments, 10 mm depth for the “prostate” compartment, 0.5 mm diameter for A1 and 0.8 diameter for A2.

Here the shape of the scan curve shows an additional feature, whose magnitude depends on the ratio R. One can observe a peak at depth of about 2 mm. This peak results from a rather complex combination of the focal volume size and shape, the ratio of Zn in the two compartments, the thickness of the first compartment and the self-absorption effect.

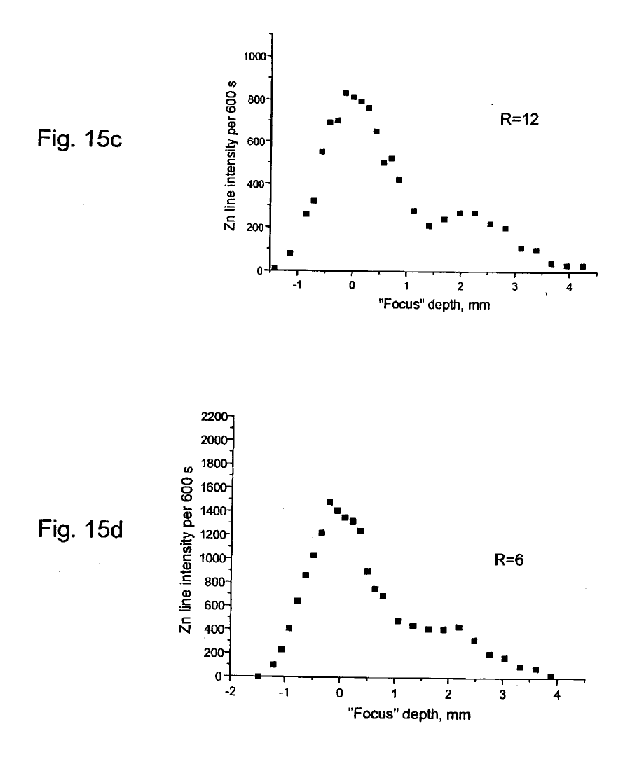

FIG. 16 shows the ratio of fluorescent intensities at depth of 2 mm to that obtained from the surface for the above scans (vertical axis) vs. R (horizontal axis). One can observe that there is quite a good linear relationship between the two ratios. This is an important observation since such a relationship could in principle be used for the estimation of Zn content in the prostate tissue just behind the rectal wall.

One should remember that the intrinsic resolution of the system in this configuration is limited, and is about 1 mm. Additional information on the position of the focal volume within the object can be obtained from the ratio of the intensities of ZnK_{α} and ZnK_{β} lines. The attenuation coefficient in water for 8.6 keV and 9.6 keV is 8.3 and 5.6 cm⁻¹ respectively. Due to the self-absorption, the ratio of intensities of the two lines will decrease with increasing depth of the focal volume.

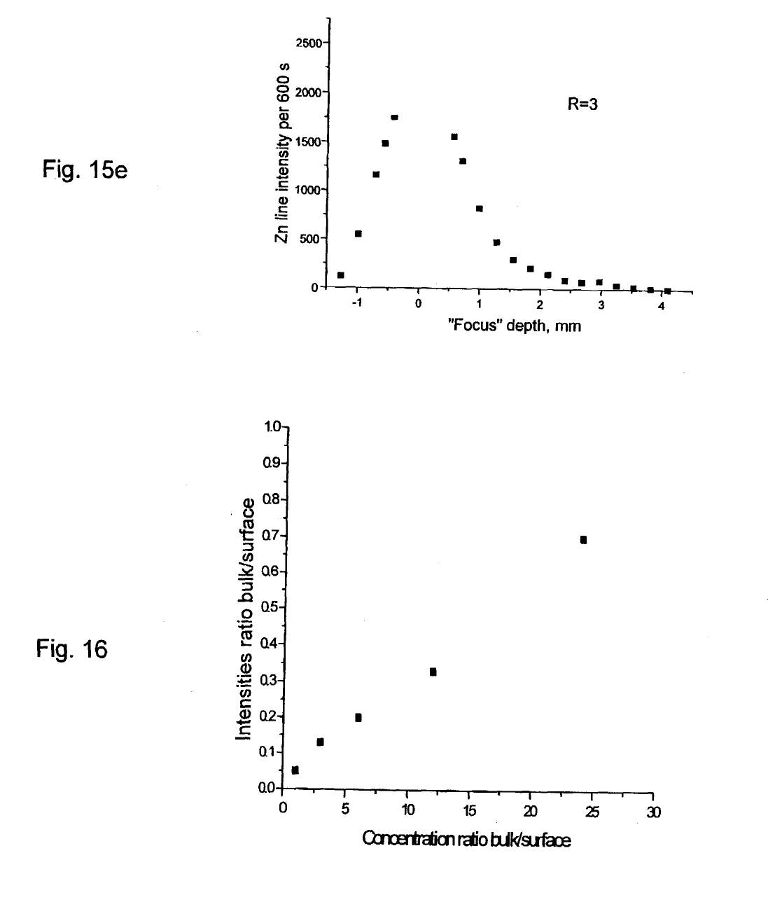

FIG. 17 shows the ratio of the K_{α} to K_{β} intensities in the uniform phantom (vertical axis) vs. the depth in the phantom (horizontal axis). It can be observed that the ratio changes rapidly, with a slope of approximately −1.5 mm⁻¹.

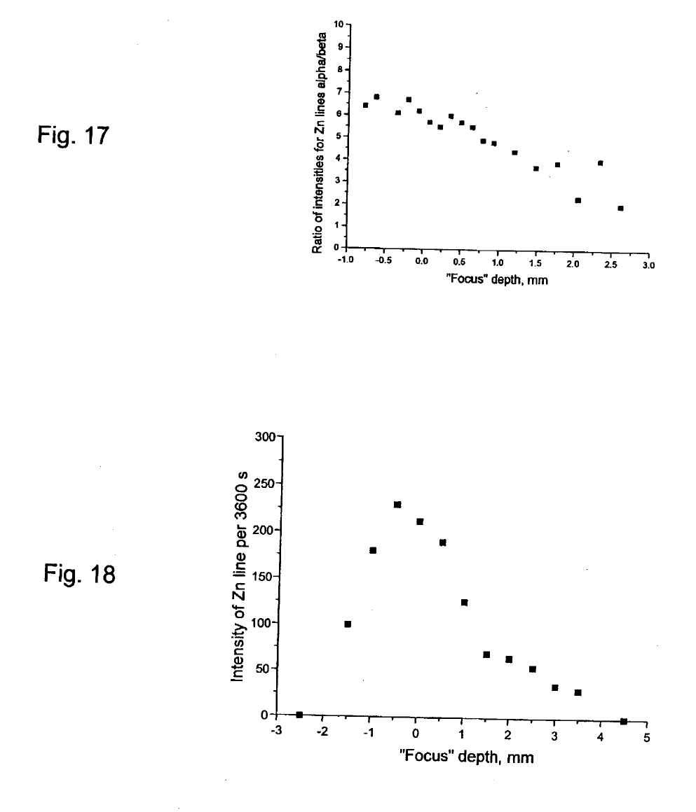

FIG. 18 shows results of scanning measurements performed at scattering angle of 150°, for R=4. As shown in FIG. 18, a peak is observed at about a depth of 2 mm. The ratio of the intensity at 2 mm depth to that at the surface was approximately 0.3. This ratio is different from the one inferred from FIG. 17 for identical R. This could be due to different focal volume shape and different self-absorption of the fluorescence radiation in the tissue equivalent phantom with smaller Zn concentration than in the aqueous phantoms.

Analysis of the Results

The shape of the scan curve can be represented as a convolution of the response function (or the point spread function) of the system with the spatial Zn distribution within the phantom and the attenuation function of the incident and exiting X-rays. The response function used here is the curve resulting from a scan of the thin Cu foil. By first treating the data from a uniform phantom one can determine parameters, which are common to all phantoms, namely, proportionality constant and an attenuation coefficient in one of the compartments. These parameters, determined by a weighted least squares (LS) method, are then used for processing, by weighted LS of all other cases. Table 3 shows the results of this analysis. The reconstructed parameters are: Zn concentration ratio, R, thickness of the first compartment, a, and a displacement of the real center of the focal volume from its assumed position foc.

TABLE 3

| Real R | Reconstructed R | a (mm) | foc (mm) |
|---|---|---|---|
| 1 | 1.040 ± 0.489 | 1.62 ± 10.65 | 0.30 ± 4.96 |
| 3 | 3.998 ± 1.225 | 2.58 ± 0.64 | 0.46 ± 0.164 |
| 6 | 6.860 ± 0.681 | 2.18 ± 0.104 | 0.43 ± 0.027 |
| 12 | 11.830 ± 0.568 | 2.44 ± 0.043 | 0.4 ± 0.007 |
| 24 | 23.165 ± 3.237 | 2.38 ± 0.075 | 0.52 ± 0.012 |

The first row in Table 3 represents results obtained from reanalyzing the uniform phantom, but treating it this time as two compartments phantom. Obviously in this case the thickness of the first compartment has no meaning. One can observe that the precision of all the reconstructed parameters improves with the ratio R.

Discussion

The phantom studies indicate that in principle it is possible to measure concentration of Zn in the prostate through the rectal wall using the described topography procedure. The spatial resolution is dictated by the size and orientation of the incident and exiting beams. The attenuation of the Zn-K_{α} 8.6 keV fluorescence line in tissue is the major factor that limits the maximal depth of the measurement. As can be observed from FIGS. 15a-e the second peak which appears at a depth of about 2 mm is smaller than the peak at the surface even at R=24. As the ratio decreases the second peak becomes smaller and is barely visible at R=3. At a ratio smaller than 3 there is no peak, but rather a tale. The strong attenuation will limit the technique to depths of a few millimeters behind the rectal wall. But this is the peripheral zone of the prostate, where most malignant tumors develop.

As has been shown in FIG. 16, a simple ratio between the number of counts at a depth of about 2 mm to that on the surface shows linear relationship with R and in principle can be used for determination of Zn content in prostate for a given thickness of the rectal wall. In practice, this approach will be limited by the statistics of the number of counts collected from the depth of the tissue. The ratio of K_{α} to K_{β} intensities vs. depth (FIG. 17) can in principle provide the information about the depth of the measurement; but again, the statistics of the K_{β} radiation will limit the usefulness of this additional information. In this example, the focal volume dimensions are of 2-3 millimeters, and there are some interference from the Zn in the “rectal wall” with that measured from the “prostate”; a more complex reconstruction technique, which makes use of several measurement points, is required. Indeed, Table 3 shows that it is possible to reconstruct R quite well even for R approaching 1 and that the precision increases with increase in R. It would appear that a statistics of about 100 counts from depth of 3 mm would be sufficient for a reliable reconstruction. Determination of the thickness of the rectal wall by other means such as Transrectal Ultrasonic Probe (TRUS) will reduce the number of reconstructed variables and will increase the precision.

A strategy for in-vivo transrectal Zn measurement may be as following: (a) The thickness of the rectal wall is determined using TRUS, (b) A measurement of Zn concentration is performed on the surface of the rectal wall and (c) at least one measurement is performed at depths behind the rectal wall. The ratio between Zn in prostate surface to that in the rectal wall can now be determined using an appropriate reconstruction algorithm for the particular wall thickness. The absolute Zn concentration in the prostate can be thus obtained by comparison with that of the rectal wall that can be determined with relatively small interference from the prostate.

Obviously the quality of the reconstruction result increases with the number of measurements in depth. However, one has to consider the radiation dose delivered to the rectal wall in each measurement. The estimated radiation exposure to the phantoms in the above experiments was 6 Röentgens, resulting from the fact that in this setup the solid angle seen by the collimated detector was very small.

In the in-vivo probe a large area detector and collimator are preferably used, with an axial symmetry around the primary incident beam. With a suitable detector and an optimized irradiation setup one can collect the required statistics of 100 counts from depth of 3 ml in less than 10 seconds with an exposure of 0.3 Röentgens.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An apparatus for non-invasive in vivo detection of a chemical element in the prostate of a subject, comprising:
(a) a probe adapted for being inserted into at least one of the rectum or the urethra of the subject;
(b) an irradiation system capable of exciting the chemical element to emit radiation to form emitted radiation;
(c) a radiation detector located within said probe, wherein said radiation detector is capable of detecting said emitted radiation and wherein said radiation detector is suitable for mapping said emitted radiation; and
(d) a signal recording, processing and displaying system for mapping the level of the chemical element in the prostate of the subject at a plurality of different points in the prostate according to said mapping of said emitted radiation.

2. The apparatus of claim 1, wherein said emitted radiation comprises fluorescent X-ray radiation.

3. The apparatus of claim 1, wherein said irradiation system is capable of delivering exciting radiation through said probe to the prostate.

4. The apparatus of claim 1, wherein said radiation detector comprises at least one of a high energy-resolution solid state detector and a high energy-resolution gaseous detector.

5. The apparatus of claim 1, wherein said radiation detector comprises at least one of a stationary detector, a scanning detector, a position-sensitive detector or an array of detectors or a combination thereof.

6. The apparatus of claim 1, wherein said radiation detector is selected from the group consisting of a radiation detector having a single element, a radiation detector having a pixelized array and a radiation detector having an array assembled of a plurality of individual elements.

7. The apparatus of claim 4, wherein said high energy-resolution solid state detector is selected from the group consisting of Silicon radiation detector, Germanium radiation detector, Silicon-Lithium-drifted radiation detector, Germanium-Lithium-drifted radiation detector, Mercury Iodide radiation detector and Cadmium-Zinc Telluride radiation detector.

8. The apparatus of claim 4, wherein said high energy-resolution gaseous detector is selected from the group consisting of a gas proportional detector and gas scintillation detector.

9. The apparatus of claim 7, wherein said high energy-resolution solid state detector is selected from the group consisting of a PIN diode, a surface barrier diode, a drift diode, a micro-strip detector, a drift chamber, a multi-pixel detector and a multi-strip detector.

10. The apparatus of claim 1, wherein said irradiation system comprises a scanning irradiation system.

11. The apparatus of claim 1, wherein said radiation detector is capable of detecting radiation from a plurality of predetermined angles so as to allow said signal recording, processing and displaying system to map said level of the chemical element at said plurality of different points.

12. The apparatus of claim 1, further comprising an arrangement of radiation detectors for detecting radiation from a plurality of predetermined angles so as to allow said signal recording, processing and displaying system to map said level of the chemical element at said plurality of different points.

13. The apparatus of claim 1, wherein the chemical element comprises zinc, wherein said radiation detector and said irradiation system are suitable for measuring the level of zinc, and wherein said signal recording, processing and displaying system maps the level of zinc to detect a possible cancer in at least a portion of the prostate.

14. The apparatus of claim 1, wherein said radiation detector is suitable for measuring the level of at least one radioactive substance introduced into the prostate.

15. The apparatus of claim 1, wherein said signal recording, processing and displaying system maps a boundary of a possible cancer in the prostate.

16. The apparatus of claim 15, wherein said signal recording, processing and displaying system maps said boundary according to a distribution of the chemical element in at least a region of the prostate being examined.

17. The apparatus of claim 15, wherein said boundary is at least partially determined according to a distribution of different concentrations of the chemical element within at least said region.

18. The apparatus of claim 17, wherein said distribution of said different concentrations of the chemical element is also used for staging the cancer.

19. The apparatus of claim 15, further comprising at least one additional mapping device for combining with information from said signal recording, processing and displaying system for determining said boundary.

20. The apparatus of claim 19, wherein said at least one additional mapping device is selected from the group consisting of a transrectal ultrasound probe and a magnetic-resonance-imaging probe.

21. The apparatus of claim 1, wherein the chemical element comprises a chemical element introduced into the prostate for a specific medical procedure, and wherein said signal recording, processing and displaying system maps the level of the chemical element to perform the specific medical procedure on at least a portion of the prostate.

22. The apparatus of claim 21, wherein said specific medical procedure comprises a photodynamic therapy.

23. The apparatus of claim 22, wherein the chemical element is Pd.

24. The apparatus of claim 21, wherein said radiation detector detects X-ray fluorescence.

25. The apparatus of claim 21, wherein the chemical element is introduced in either a quantitative or a qualitative amount.

26. The apparatus of claim 1, wherein the chemical element to be detected comprises one or more of Zn, Fe, Ca, Br, or Pd.

27. The apparatus of claim 1, wherein the chemical element to be detected comprises Zn.

28. The apparatus of claim 2, wherein the chemical element to be detected emits characteristic fluorescent X-rays according to an identity of the chemical element, and wherein an intensity of said characteristic fluorescent X-rays correlates to a concentration of the chemical element, such that said radiation detector is adapted to detect at least one chemical element according to said characteristic fluorescent X-rays and to measure said intensity.

29. The apparatus of claim 1, wherein said irradiation system comprises at least one of a radioactive source, an X-ray tube, a synchrotron light source, an X-ray beam guide connected to an external X-ray source or a miniature plasma X-ray generator.

30. The apparatus of claim 1, wherein said irradiation system is coupled to a monochromatizing element so as to provide a radiation with a substantially accurate energy.

31. The apparatus of claim 30, wherein said monochromatizing element is selected from the group consisting of a crystal monochromator and a plurality of different absorbing films each characterized by a different absorption coefficient.

32. The apparatus of claim 1, further comprising a biopsy device.

33. The apparatus of claim 1, further comprising a device for injection of a drug or a contrast agent.

34. The apparatus of claim 1, further comprising a device for illumination of the prostate with light.

35. The apparatus of claim 1, further comprising a normalizer for normalizing measurement of said emitted radiation according to a normalizing measurement of a reference element.

36. The apparatus of claim 35, wherein said normalizer is operable to normalize said emitted radiation according to an amount of Compton scattered radiation of radiation emitted by said irradiation system.

37. The apparatus of claim 1, wherein said radiation detector is characterized by geometry selected from the group consisting of planar geometry, spherical geometry cylindrical geometry and an irregular geometry.

38. The apparatus of claim 1, further comprising an X-ray optical system, located within said probe, wherein said X-ray optical system is selected so as to collimate and/or focus radiation emitted by said irradiation system and/or radiation emitted by the chemical element.

39. The apparatus of claim 38, wherein said X-ray optical system comprises a focusing element for focusing said radiation emitted by said irradiation system.

40. The apparatus of claim 39, wherein said focusing element is selected from the group consisting of a capillary optical device and an aperture.

41. The apparatus of claim 38, wherein said X-ray optical system comprises a collimator for collimating said radiation emitted by the chemical element into said radiation detector.

42. The apparatus of claim 38, wherein said X-ray optical system comprises a collimating element for collimating said radiation emitted by said irradiation system.

43. The apparatus of claim 38, wherein said X-ray optical system comprises a capillary X-ray optics for focusing and collimating said radiation emitted by said irradiation system.

44. The apparatus of claim 41, wherein said collimator is characterized by geometry selected from the group consisting of planar geometry, spherical geometry cylindrical geometry and an irregular geometry.

45. The apparatus of claim 1, further comprising electronic circuitry, adapted for being located within said probe, wherein said electronic circuitry is designed and constructed for transmitting signals from said radiation detector to said signal recording, processing and displaying system.

46. The apparatus of claim 1, further comprising a thermoelectric cooling system, adapted for being located within said probe, wherein said thermoelectric cooling system is designed and constructed for cooling said radiation detector to have improved energy resolution.

47. The apparatus of claim 1, wherein said radiation detector is capable of discriminating between radiation emitted by the chemical element being present in the prostate and radiation emitted by chemical elements being present in tissues surrounding said prostate, thereby to map the prostate.

48. The apparatus of claim 1, further comprising a collimator for collimating said emitted radiation in a manner that radiation emitted by chemical elements being present in tissues other than tissues of the prostate is absorbed by said collimator.

49. The apparatus of claim 1, wherein said collimator is made of a substrate having a plurality predetermined radiation paths, wherein said plurality of predetermined radiation paths is selected from the group consisting of radiation paths directing radiation emitted from the chemical element in a single location to a plurality of locations on said radiation detector, radiation paths directing the radiation emitted from the chemical element in a plurality of locations to a plurality of locations on said radiation detector, and radiation paths directing the radiation emitted from the chemical element in a plurality of locations to a plurality of detector-elements.

50. The apparatus of claim 49, wherein each of said plurality of predetermined radiation paths is selected from the group consisting of a thin aperture, a thin capillary and an X-ray optical element.

51. The apparatus of claim 1, wherein said radiation detector is capable of simultaneously detecting said emitted radiation from a plurality of depth inside the prostate.

52. The apparatus of claim 1, further comprising an arrangement of radiation detectors and a collimator, wherein said collimator is capable of collimating radiation emitted from different depths inside the prostate into different locations of said radiation detector or different radiation detectors.

53. The apparatus of claim 1, further comprising a transrectal ultrasound probe.

54. A method of non-invasive in vivo detection of a chemical element in the prostate of a subject, comprising:
endoscopically inserting a probe into the subject;
irradiating the prostate with said probe by exciting radiation thereby exciting the chemical element to emit radiation to form emitted radiation;
detecting and mapping said emitted radiation with said probe; and
mapping the level of the chemical element in the prostate of the subject at a plurality of different points in the prostate according to said mapping of said emitted radiation.

55. She method of claim 54, wherein said emitted radiation comprises fluorescent X-ray radiation.

56. The method of claim 54, wherein said endoscopically inserting said probe is into the rectum or the urethra of the subject.

57. The method of claim 54, wherein said detecting said emitted radiation is by a radiation detector which comprises at least one of a stationary detector, a scanning detector, a position-sensitive detector or an array of detectors or a combination thereof.

58. The method of claim 57, wherein said radiation detector is selected from the group consisting of a radiation detector having a single element, a radiation detector having a pixelized array and a radiation detector having an array assembled of a plurality of individual elements.

59. The method of claim 57, wherein said radiation detector comprises at least one of a high energy-resolution solid state detector and a high energy-resolution gaseous detector.

60. The method of claim 59, wherein said high energy-resolution solid state detector is selected from the group consisting of Silicon radiation detector, Germanium radiation detector, Silicon-Lithium-drifted radiation detector, Germanium-Lithium-drifted radiation detector, Mercury Iodide radiation detector and Cadmium-Zinc Telluride radiation detector.

61. The method of claim 59, wherein said high energy-resolution gaseous detector is selected from the group consisting of a gas proportional detector and gas scintillation detector.

62. The method of claim 60, wherein said high energy-resolution solid state detector is selected from the group consisting of a PIN diode, a surface barrier diode, a drift diode, a micro-strip detector, a drift chamber, a multi-pixel detector and a multi-strip detector.

63. The method of claim 54, wherein said irradiating comprises scanning the prostate so as to excite the chemical element to emit said radiation from a plurality of predetermined angles.

64. The method of claim 54, wherein said detecting said emitted radiation is by scanning the prostate so as to detect said emitted radiation from a plurality of predetermined angles.

65. The method of claim 54, wherein said detecting said emitted radiation is by an arrangement of radiation detectors arranged so as to detect said emitted radiation from a plurality of predetermined angles.

66. The method of claim 54, wherein the chemical element comprises zinc, and wherein the level of zinc is used for detecting a possible cancer in at least a portion of the prostate.

67. The method of claim 54, further comprising introducing at least one radioactive substance into the prostate and measuring the level of said at least one radioactive substance in the prostate.

68. The method of claim 66, further comprising mapping a boundary of said possible cancer in the prostate.

69. The method of claim 68, wherein said mapping said boundary is according to a distribution of the chemical element in at least a region of the prostate being examined.

70. The method of claim 68, wherein said boundary is at least partially determined according to a distribution of different concentrations of the chemical element within at least said region.

71. The method of claim 70, further comprising using said distribution of said different concentrations of the chemical element for staging the cancer.

72. The method of claim 68, further comprising mapping the prostate using at least one mapping method other than an X-ray fluorescence method and information obtained from said at least one mapping method with information from said emitted radiation for determining said boundary.

73. The method of claim 72, wherein said at least one additional mapping method is selected from the group consisting of ultrasonic imaging and a magnetic-resonance-imaging.

74. The method of claim 54, wherein the chemical element comprises a chemical element introduced into the prostate for a specific medical procedure, and wherein said mapping the level of the chemical element is used for performing the specific medical procedure on at least a portion of the prostate.

75. The method of claim 74, wherein said specific medical procedure comprises a photodynamic therapy.

76. The method of claim 75, wherein the chemical element is Pd.

77. The method of claim 74, wherein the chemical element is introduced in either a quantitative or a qualitative amount.

78. The method of claim 54, wherein the chemical element to be detected comprises one or more of Zn, Fe, Ca, Br, or Pd.

79. The method of claim 54, wherein the chemical element to be detected comprises Zn.

80. The method of claim 55, wherein the chemical element to be detected emits characteristic fluorescent X-rays according to an identity of the chemical element, and wherein an intensity of said characteristic fluorescent X-rays correlates to a concentration of the chemical element, such that said radiation detector is adapted to detect at least one chemical element according to said characteristic fluorescent X-rays and to measure said intensity.

81. The method of claim 54, wherein said irradiating the prostate is by an irradiation system comprising at least one of a radioactive source, an X-ray tube, a synchrotron light source, an X-ray beam guide connected to an external X-ray source or a miniature plasma X-ray generator.

82. The method of claim 54, wherein said irradiation system is coupled to a monochromatizing element so as to provide a radiation with a substantially accurate energy.

83. The method of claim 82, wherein said monochromatizing element is selected from the group consisting of a crystal monochromator and a plurality of different absorbing films each characterized by a different absorption coefficient.

84. The method of claim 54, further comprising using said probe for performing a biopsy procedure.

85. The method of claim 54, further comprising using said probe for injection of a drug or a contrast agent into the prostate.

86. The method of claim 54, further comprising using said probe for illuminating the prostate with light.

87. The method of claim 54, further comprising a normalizing measurement of said emitted radiation according to a normalizing measurement of a reference element.

88. The apparatus of claim 87, wherein said normalizing is according to an amount of Compton scattered radiation of radiation emitted by said irradiation system

89. The method of claim 54, further comprising collimating and focusing said exciting radiation and said emitted radiation.

90. The method of claim 54, further comprising imaging the prostate using a transrectal ultrasound probe.

91. The method of claim 57, further comprising cooling said radiation detector to have improved energy resolution.

92. The method of claim 91, wherein said cooling said radiation detector is by a thermoelectric cooling system, adapted for being located within said probe.

93. The method of claim 54, further comprising discriminating between radiation emitted by the chemical element being present in the prostate and radiation emitted by chemical elements being present in tissues surrounding said prostate, thereby to map the prostate.

94. The method of claim 54, further collimating said emitted radiation in a manner that radiation emitted by chemical elements being present in tissues other than tissues of the prostate is absorbed.

95. The method of claim 54, further comprising simultaneously detecting said emitted radiation from a plurality of depth inside the prostate.

96. The method of claim 54, further comprising collimating radiation emitted from different depths inside the prostate into different locations of a radiation detector or different radiation detectors.

97. A system for diagnosing prostate cancer in the prostate of a subject, the system comprising:
(a) a first apparatus for determining a first parameter being a level of a chemical element in the prostate;
(b) a second apparatus for determining a second parameter being indicative of prostate specific antigen (PSA) activity in the blood serum of the subject; and
(c) a data processor programmed to diagnose the prostate cancer if said first parameter has a predetermined relation with respect to a first predetermined threshold and said second parameter has a predetermined relation with respect to a second predetermined threshold.

98. The system of claim 97, wherein said predetermined relation of each of said first and said second parameters is independently selected from the group consisting of above and below a respective predetermined threshold.

99. The system of claim 97, wherein said first apparatus is operable to detect said first level of said chemical element in vivo or in vitro.

100. The system of claim 97, wherein said second parameter is selected from the group consisting of serum PSA level, PSA density, PSA velocity, a level of age specific PSA, and percentage of free PSA.

101. The system of claim 97, wherein said first apparatus is an X-ray fluorescence-based apparatus.

102. The system of claim 97, wherein said second apparatus selected from the group consisting of an activation analysis-base apparatus, an atomic absorption-based apparatus, and a particle-induced X-ray emission-based apparatus.

103. The system of claim 97, wherein said chemical element comprises Zn.

104. The system of claim 97, farther comprising a biopsy device.

105. The system of claim 97, wherein said first apparatus comprises:
(i) a probe adapted for being inserted into at least one of the rectum or the urethra of the subject;
(ii) an irradiation system capable of exciting said chemical element to emit radiation to form emitted radiation; and
(iii) a radiation detector located within said probe, wherein said radiation detector is capable of detecting said emitted radiation and wherein said radiation detector is suitable for mapping said emitted radiation.

106. The system of claim 105, wherein said emitted radiation comprises fluorescent X-ray radiation.

107. The system of claim 105, wherein said irradiation system is capable of delivering exciting radiation through said probe to the prostate.

108. The system of claim 105, further comprising a signal recording, processing and displaying system electrically communicating with said data processor, and operable to map said level of said chemical element at a plurality of different points in the prostate according to said mapping of said emitted radiation.

109. The system of claim 105, wherein said radiation detector comprises at least one of a high energy-resolution solid state detector and a high energy-resolution gaseous detector.

110. The system of claim 105, wherein said radiation detector is selected from the group consisting of a radiation detector having a single element, a radiation detector having a pixelized array and a radiation detector having an array assembled of a plurality of individual elements.

111. The system of claim 105, wherein said radiation detector comprises at least one of a stationary detector, a scanning detector, a position-sensitive detector or an array of detectors or a combination thereof.

112. The system of claim 109, wherein said high energy-resolution gaseous detector is selected from the group consisting of a gas proportional detector and gas scintillation detector.

113. The system of claim 109, wherein said high energy-resolution solid state detector is selected from the group consisting of Silicon radiation detector, Germanium radiation detector, Silicon-Lithium-drifted radiation detector, Germanium-Lithium-drifted radiation detector, Mercury Iodide radiation detector and Cadmium-Zinc Telluride radiation detector.

114. The system of claim 113, wherein said high energy-resolution solid state detector is selected from the group consisting of a PIN diode, a surface barrier diode, a drift diode, a micro-strip detector, a drift chamber, a multi-pixel detector and a multi-strip detector.

115. The system of claim 105, wherein said irradiation system comprises a scanning irradiation system.

116. The system of claim 108, wherein said radiation detector is capable of detecting radiation from a plurality of predetermined angles so as to allow said signal recording, processing and displaying system to map said level of said chemical element at said plurality of different points.

117. The system of claim 108, further comprising an arrangement of radiation detectors for detecting radiation from a plurality of predetermined angles so as to allow said signal recording, processing and displaying system to map said level of said chemical element at said plurality of different points.

118. The system of claim 105, wherein said radiation detector is suitable for measuring the level of at least one radioactive substance introduced into the prostate.

119. The system of claim 105, wherein said signal recording, processing and displaying system maps a boundary of the prostate cancer in the prostate.

120. The system of claim 119, wherein said signal recording, processing and displaying system maps said boundary according to a distribution of said chemical element in at least a region of the prostate being examined.

121. The system of claim 119, wherein said boundary is at least partially determined according to a distribution of different concentrations of said chemical element within at least said region.

122. The system of claim 121, wherein said distribution of said different concentrations of said chemical element is also used for staging the cancer.

123. The system of claim 108, further comprising at least one additional mapping device for combining with information from said signal recording, processing and displaying system for determining said boundary.

124. The system of claim 118, wherein said at least one additional mapping device is selected from the group consisting of a transrectal ultrasound probe and a magnetic-resonance-imaging probe.

125. The system of claim 108, wherein said chemical element comprises a chemical element introduced into the prostate for a specific medical procedure, and wherein said signal recording, processing and displaying system maps the level of said chemical element to perform the specific medical procedure on at least a portion of the prostate.

126. The system of claim 125, wherein said specific medical procedure comprises a photodynamic therapy.

127. The system of claim 126, wherein the chemical element is Pd.

128. The system of claim 125, wherein said radiation detector detects X-ray fluorescence.

129. The system of claim 125, wherein said chemical element is introduced in either a quantitative or a qualitative amount.

130. The system of claim 105, wherein said chemical element to be detected comprises one or more of Zn, Fe, Ca, Br, or Pd.

131. The system of claim 106, wherein said chemical element to be detected emits characteristic fluorescent X-rays according to an identity of said chemical element, and wherein an intensity of said characteristic fluorescent X-rays correlates to a concentration of said chemical element, such that said radiation detector is adapted to detect at least one chemical element according to said characteristic fluorescent X-rays and to measure said intensity.

132. The system of claim 105, wherein said irradiation system comprises at least one of a radioactive source, an X-ray tube, a synchrotron light source, an X-ray beam guide connected to an external X-ray source or a miniature plasma X-ray generator.

133. The system of claim 105, wherein said irradiation system is coupled to a monochromatizing element so as to provide a radiation with a substantially accurate energy.

134. The system of claim 133, wherein said monochromatizing element is selected from the group consisting of a crystal monochromator and a plurality of different absorbing films each characterized by a different absorption coefficient.

135. The system of claim 105, further comprising a device for injection of a drug or a contrast agent.

136. The system of claim 105, further comprising a device for illumination of the prostate with light.

137. The system of claim 105, further comprising a normalizer for normalizing measurement of said emitted radiation according to a normalizing measurement of a reference element.

138. The system of claim 137, wherein said normalizer is operable to normalize said emitted radiation according to an amount of Compton scattered radiation of radiation emitted by said irradiation system.

139. The system of claim 105, wherein said radiation detector is characterized by geometry selected from the group consisting of planar geometry, spherical geometry cylindrical geometry and an irregular geometry.

140. The system of claim 105, further comprising an X-ray optical system, located within said probe, wherein said X-ray optical system is selected so as to collimate and/or focus radiation emitted by said irradiation system and/or radiation emitted by said chemical element.

141. The system of claim 140, wherein said X-ray optical system comprises a focusing element for focusing said radiation emitted by said irradiation system.

142. The system of claim 141, wherein said focusing element is selected from the group consisting of a capillary optical device and an aperture.

143. The system of claim 140, wherein said X-ray optical system comprises a collimating element for collimating said radiation emitted by said irradiation system

144. The system of claim 140, wherein said X-ray optical system comprises a capillary X-ray optics for focusing and collimating said radiation emitted by said irradiation system.

145. The system of claim 140, wherein said X-ray optical system comprises a collimator for collimating said radiation emitted by said chemical element into said radiation detector.

146. The system of claim 145, wherein said collimator is characterized by geometry selected from the group consisting of planar geometry, spherical geometry cylindrical geometry and an irregular geometry.

147. The system of claim 108, further comprising electronic circuitry, adapted for being located within said probe, wherein said electronic circuitry is designed and constructed for transmitting signals from said radiation detector to said signal recording, processing and displaying system.

148. The system of claim 105, further comprising a thermoelectric cooling system, adapted for being located within said probe, wherein said thermoelectric cooling system is designed and constructed for cooling said radiation detector to have improved energy resolution.

149. The system of claim 105, wherein said radiation detector is capable of discriminating between radiation emitted by said chemical element being present in the prostate and radiation emitted by chemical elements being present in tissues surrounding said prostate, thereby to map the prostate.

150. The system of claim 105, further comprising a collimator for collimating said emitted radiation in a manner that radiation emitted by chemical elements being present in tissues other than tissues of the prostate is absorbed by said collimator.

151. The system of claim 150, wherein said collimator is made of a substrate having a plurality predetermined radiation paths, wherein said plurality of predetermined radiation paths is selected from the group consisting of radiation paths directing radiation emitted from the chemical element in a single location to a plurality of locations on said radiation detector, radiation paths directing the radiation emitted from the chemical element in a plurality of locations to a plurality of locations on said radiation detector, and radiation paths directing the radiation emitted from the chemical element in a plurality of locations to a plurality of detector-elements.

152. The system of claim 150, wherein each of said plurality of predetermined radiation paths is selected from the group consisting of a thin aperture, a thin capillary and an X-ray optical element.

153. The system of claim 105, wherein said radiation detector is capable of simultaneously detecting said emitted radiation from a plurality of depth inside the prostate.

154. The system of claim 105, further comprising an arrangement of radiation detectors and a collimator, wherein said collimator is capable of collimating radiation emitted from different depths inside the prostate into different locations of said radiation detector or different radiation detectors.

155. The system of claim 105, further comprising a transrectal ultrasound probe.

156. A method of diagnosing prostate cancer in the prostate of a subject, the method comprising:
determining a first parameter being a level of a chemical element in the prostate;
determining a second parameter being indicative of prostate specific antigen (PSA) activity in the blood serum of the subject; and
wherein the prostate cancer is diagnosed if said first parameter has a predetermined relation with respect to a first predetermined threshold and said second parameter has a predetermined relation with respect to a second predetermined threshold.

157. The method of claim 156, wherein said predetermined relation of each of said first and said second parameters is independently selected from the group consisting of above and below a respective predetermined threshold.

158. The method of claim 156, wherein said determining said level of said chemical element is done in vivo or in vitro.

159. The method of claim 156, wherein said second parameter is selected from the group consisting of serum PSA level, PSA density, PSA velocity, a level of age specific PSA, and percentage of free PSA.

160. The method of claim 156, wherein said determining said level of said chemical element is by X-ray fluorescence.

161. The method of claim 156, wherein said determining said level of said chemical element is affected by a procedure selected from the group consisting of an activation analysis, an atomic absorption a particle-induced X-ray emission.

162. The method of claim 156, wherein said chemical element comprises Zn.

163. The method of claim 156, wherein said determining said level of said chemical element comprises:
endoscopically inserting a probe into the subject;
irradiating the prostate with said probe by exciting radiation thereby exciting said chemical element to emit radiation to form emitted radiation;
detecting and mapping said emitted radiation with said probe; and
mapping the level of said chemical element in the prostate of the subject at a plurality of different points in the prostate according to said mapping of said emitted radiation.

164. The method of claim 163, wherein said emitted radiation comprises fluorescent X-ray radiation.

165. The method of claim 163, wherein said endoscopically inserting said probe is into the rectum or the urethra of the subject.

166. The method of claim 163, wherein said detecting said emitted radiation is by a radiation detector which comprises at least one of a stationary detector, a scanning detector, a position-sensitive detector or an array of detectors or a combination thereof.

167. The method of claim 166, wherein said radiation detector is selected from the group consisting of a radiation detector having a single element, a radiation detector having a pixelized array and a radiation detector having an array assembled of a plurality of individual elements.

168. The method of claim 166, wherein said radiation detector comprises at least one of a high energy-resolution solid state detector and a high energy-resolution gaseous detector.

169. The method of claim 168, wherein said high energy-resolution solid state detector is selected from the group consisting of Silicon radiation detector, Germanium radiation detector, Silicon-Lithium-drifted radiation detector, Germanium-Lithium-drifted radiation detector, Mercury Iodide radiation detector and Cadmium-Zinc Telluride radiation detector.

170. The method of claim 168, wherein said high energy-resolution gaseous detector is selected from the group consisting of a gas proportional detector and gas scintillation detector.

171. The method of claim 169, wherein said high energy-resolution solid state detector is selected from the group consisting of a PIN diode, a surface barrier diode, a drift diode, a micro-strip detector, a drift chamber, a multi-pixel detector and a multi-strip detector.

172. The method of claim 164, wherein said irradiating comprises scanning the prostate so as to excite said chemical element to emit said fluorescent X-ray radiation from a plurality of predetermined angles.

173. The method of claim 163, wherein said detecting said emitted radiation is by scanning the prostate so as to detect said emitted radiation from a plurality of predetermined angles.

174. The method of claim 163, wherein said detecting said emitted radiation is by an arrangement of radiation detectors arranged so as to detect said emitted radiation from a plurality of predetermined angles.

175. The method of claim 163, further comprising introducing at least one radioactive substance into the prostate and measuring the level of said at least one radioactive substance in the prostate.

176. The method of claim 163, further comprising mapping a boundary of the prostate cancer in the prostate.

177. The method of claim 176, wherein said mapping said boundary is according to a distribution of said chemical element in at least a region of the prostate being examined.

178. The method of claim 176, wherein said boundary is at least partially determined according to a distribution of different concentrations of said chemical element within at least said region.

179. The method of claim 178, further comprising using said distribution of said different concentrations of said chemical element for staging the cancer.

180. The method of claim 176, further comprising mapping the prostate using at least one mapping method other than an X-ray fluorescence method and information obtained from said at least one mapping method with information from said emitted radiation for determining said boundary.

181. The method of claim 180, wherein said at least one additional mapping method is selected from the group consisting of ultrasonic imaging and a magnetic-resonance-imaging.

182. The method of claim 163, wherein said chemical element comprises a chemical element introduced into the prostate for a specific medical procedure, and wherein said mapping the level of said chemical element is used for performing the specific medical procedure on at least a portion of the prostate.

183. The method of claim 182, wherein said specific medical procedure comprises a photodynamic therapy.

184. The method of claim 182, wherein the chemical element is Pd.

185. The method of claim 182, wherein said chemical element is introduced in either a quantitative or a qualitative amount.

186. The method of claim 163, wherein said chemical element to be detected comprises one or more of Zn, Fe, Ca, Br, or Pd.

187. The method of claim 164, wherein said chemical element to be detected emits characteristic fluorescent X-rays according to an identity of said chemical element, and wherein an intensity of said characteristic fluorescent X-rays correlates to a concentration of said chemical element, such that said radiation detector is adapted to detect at least one chemical element according to said characteristic fluorescent X-rays and to measure said intensity.

188. The method of claim 163, wherein said irradiating the prostate is by an irradiation system comprising at least one of a radioactive source, an X-ray tube, a synchrotron light source, an X-ray beam guide connected to an external X-ray source or a miniature plasma X-ray generator.

189. The method of claim 163, wherein said irradiation system is coupled to a monochromatizing element so as to provide a radiation with a substantially accurate energy.

190. The method of claim 189, wherein said monochromatizing element is selected from the group consisting of a crystal monochromator and a plurality of different absorbing films each characterized by a different absorption coefficient.

191. The method of claim 163, further comprising using said probe for performing a biopsy procedure.

192. The method of claim 163, further comprising using said probe for injection of a drug or a contrast agent into the prostate.

193. The method of claim 163, further comprising using said probe for illuminating the prostate with light.

194. The method of claim 163, farther comprising a normalizing measurement of said emitted radiation according to a normalizing measurement of a reference element.

195. The method of claim 194, wherein said normalizing is according to an amount of Compton scattered radiation of radiation emitted by said irradiation system

196. The method of claim 163, further comprising collimating and focusing said exciting radiation and said emitted radiation.

197. The method of claim 163, further comprising imaging the prostate using a transrectal ultrasound probe.

198. The method of claim 166, further comprising cooling said radiation detector to have improved energy resolution.

199. The method of claim 198, wherein said cooling said radiation detector is by a thermoelectric cooling system, adapted for being located within said probe.

200. The method of claim 163, further comprising discriminating between radiation emitted by said chemical element being present in the prostate and radiation emitted by chemical elements being present in tissues surrounding said prostate, thereby to map the prostate.

201. The method of claim 163, further collimating said emitted radiation in a manner that radiation emitted by chemical elements being present in tissues other than tissues of the prostate is absorbed.

202. The method of claim 163, further comprising simultaneously detecting said emitted radiation from a plurality of depth inside the prostate.

203. The method of claim 163, further comprising collimating radiation emitted from different depths inside the prostate into different locations of a radiation detector or different radiation detectors.

204. A system for mapping a prostate of a subject, the system comprising:
(a) at least one mapping device;
(b) an irradiation system capable of exciting a chemical element in the prostate to emit radiation to form emitted radiation;
(c) an endoscopic probe for detecting said chemical element, said endoscopic probe comprises a radiation detector capable of detecting said emitted radiation and suitable for mapping said emitted radiation; and
(d) a data processor for mapping the prostate according to information collected from said at least one mapping device and said endoscopic probe.

205. The apparatus of claim 204, wherein said emitted radiation comprises fluorescent X-ray radiation.

206. The system of claim 204, wherein said irradiation system is capable of delivering exciting radiation through said probe to the prostate.

207. The system of claim 204, wherein said at least one mapping device is selected from the group consisting of an ultrasonic device, a magnetic-resonance-imaging device and a computer tomography device.

208. The system of claim 204, wherein said at least one mapping device is endoscopic.

209. The system of claim 204, wherein said data processor comprises a signal recording, processing and displaying system for mapping the level of said chemical element in the prostate of the subject at a plurality of different points in the prostate according to said mapping of said emitted radiation.

210. The system of claim 204, wherein said radiation detector comprises at least one of a high energy-resolution solid state detector and a high energy-resolution gaseous detector.

211. The system of claim 204, wherein said radiation detector is selected from the group consisting of a radiation detector having a single element, a radiation detector having a pixelized array and a radiation detector having an array assembled of a plurality of individual elements.

212. The system of claim 204, wherein said radiation detector comprises at least one of a stationary detector, a scanning detector, a position-sensitive detector or an array of detectors or a combination thereof.

213. The system of claim 210, wherein said high energy-resolution gaseous detector is selected from the group consisting of a gas proportional detector and gas scintillation detector.

214. The system of claim 210, wherein said high energy-resolution solid state detector is selected from the group consisting of Silicon radiation detector, Germanium radiation detector, Silicon-Lithium-drifted radiation detector, Germanium-Lithium-drifted radiation detector, Mercury Iodide radiation detector and Cadmium-Zinc Telluride radiation detector.

215. The system of claim 214, wherein said high energy-resolution solid state detector is selected from the group consisting of a PIN diode, a surface barrier diode, a drift diode, a micro-strip detector, a drift chamber, a multi-pixel detector and a multi-strip detector.

216. The system of claim 204, wherein said irradiation system comprises a scanning irradiation system.

217. The system of claim 209, wherein said radiation detector is capable or detecting radiation from a plurality of predetermined angles so as to allow said signal recording, processing and displaying system to map said level of said chemical element at said plurality of different points.

218. The system of claim 209, further comprising an arrangement of radiation detectors for detecting radiation from a plurality of predetermined angles so as to allow said signal recording, processing and displaying system to map said level of said chemical element at said plurality of different points.

219. The system of claim 209, wherein said chemical element comprises zinc, wherein said radiation detector and said irradiation system are suitable for measuring the level of zinc, and wherein said signal recording, processing and displaying system maps the level of zinc to detect a possible cancer in at least a portion of the prostate.

220. The system of claim 204, wherein said radiation detector is suitable for measuring the level of at least one radioactive substance introduced into the prostate.

221. The system of claim 219, wherein said signal recording, processing and displaying system maps a boundary of possible cancer in the prostate.

222. The system of claim 221, wherein said signal recording, processing and displaying system maps said boundary according to a distribution of said chemical element in at least a region of the prostate being examined.

223. The system of claim 221, wherein said boundary is at least partially determined according to a distribution of different concentrations of said chemical element within at least said region.

224. The system of claim 223, wherein said distribution of said different concentrations of said chemical element is also used for staging the cancer.

225. The system of claim 209, wherein said chemical element comprises a chemical element introduced into the prostate for a specific medical procedure, and wherein said signal recording, processing and displaying system maps the level of said chemical element to perform the specific medical procedure on at least a portion of the prostate.

226. The system of claim 225, wherein said specific medical procedure comprises a photodynamic therapy.

227. The system of claim 226, wherein the chemical element is Pd.

228. The system of claim 225, wherein said radiation detector detects X-ray fluorescence.

229. The system of claim 225, wherein said chemical element is introduced in either a quantitative or a qualitative amount.

230. The system of claim 204, wherein said chemical element to be detected comprises one or more of Zn, Fe, Ca, Br, or Pd.

231. The system of claim 204, wherein said chemical element to be detected comprises Zn.

232. The system of claim 205, wherein said chemical element to be detected emits characteristic fluorescent X-rays according to an identity of said chemical element, and wherein an intensity of said characteristic fluorescent X-rays correlates to a concentration of said chemical element, such that said radiation detector is adapted to detect at least one chemical element according to said characteristic fluorescent X-rays and to measure said intensity.

233. The system of claim 204, wherein said irradiation system comprises at least one of a radioactive source, an X-ray tube, a synchrotron light source, an X-ray begun guide connected to an external X-ray source or a miniature plasma X-ray generator.

234. The system of claim 204, wherein said irradiation system is coupled to a monochromatizing element so as to provide a radiation with a substantially accurate energy.

235. The system of claim 234, wherein said monochromatizing element is selected from the group consisting of a crystal monochromator and a plurality of different absorbing films each characterized by a different absorption coefficient.

236. The system of claim 204, further comprising a biopsy device.

237. The system of claim 204, further comprising a device for injection of a drug or a contrast agent.

238. The system of claim 204, further comprising a device for illumination of the prostate with light.

239. The system of claim 204, further comprising a normalizer for normalizing measurement of said emitted radiation according to a normalizing measurement of a reference element.

240. The system of claim 239, wherein said normalizer is operable to normalize said emitted radiation according to an amount of Compton scattered radiation of radiation emitted by said irradiation system.

241. The system of claim 204, wherein said radiation detector is characterized by geometry selected from the group consisting of planar geometry, spherical geometry cylindrical geometry and an irregular geometry.

242. The system of claim 204, further comprising an X-ray optical system, located within said probe, wherein said X-ray optical system is selected so as to collimate and/or focus radiation emitted by said irradiation system and/or radiation emitted by said chemical element.

243. The system of claim 242, wherein said X-ray optical system comprises a focusing element for focusing said radiation emitted by said irradiation system.

244. The system of claim 243, wherein said focusing element is selected from the group consisting of a capillary optical device and an aperture.

245. The apparatus of claim 242, wherein said X-ray optical system comprises a collimating element for collimating said radiation emitted by said irradiation system.

246. The apparatus of claim 242, wherein said X-ray optical system comprises a capillary X-ray optics for focusing arid collimating said radiation emitted by said irradiation system.

247. The system of claim 242, wherein said X-ray optical system comprises a collimator for collimating said radiation emitted by said chemical element into said radiation detector.

248. The system of claim 247, wherein said collimator is characterized by geometry selected from the group consisting of planar geometry, spherical geometry cylindrical geometry and an irregular geometry.

249. The system of claim 204, further comprising electronic circuitry, adapted for being located within said probe, wherein said electronic circuitry is designed and constructed for transmitting signals from said radiation detector to said signal recording, processing and displaying system.

250. The system of claim 204, further comprising a thermoelectric cooling system, adapted for being located within said probe, wherein said thermoelectric cooling system is designed and constructed for cooling said radiation detector to have improved energy resolution.

251. The system of claim 204, wherein said radiation detector is capable of discriminating between radiation emitted by said chemical element being present in the prostate and radiation emitted by chemical elements being present in tissues surrounding said prostate, thereby to map the prostate.

252. The system of claim 204, further comprising a collimator for collimating said emitted radiation in a manner that radiation emitted by chemical elements being present in tissues other than tissues of the prostate is absorbed by said collimator.

253. The system of claim 252, wherein said collimator is made of a substrate having a plurality predetermined radiation paths, wherein said plurality of predetermined radiation paths is selected from the group consisting of radiation paths directing radiation emitted from the chemical element in a single location to a plurality of locations on said radiation detector, radiation paths directing the radiation emitted from the chemical element in a plurality of locations to a plurality of locations on said radiation detector, and radiation paths directing the radiation emitted from the chemical element in a plurality of locations to a plurality of detector-elements.

254. The system of claim 253, wherein each of said plurality of predetermined radiation paths is selected from the group consisting of a thin aperture, a thin capillary and an X-ray optical element.

255. The system of claim 204, wherein said radiation detector is capable of simultaneously detecting said emitted radiation from a plurality of depth inside the prostate.

256. The system of claim 204, further comprising an arrangement of radiation detectors and a collimator, wherein said collimator is capable of collimating radiation emitted from different depths inside the prostate into different locations of said radiation detector or different radiation detectors.

257. A method of mapping a prostate of a subject, the method comprising:
endoscopically insetting a probe into the subject;
using said probe for irradiating the prostate by exciting radiation thereby exciting said chemical element to emit radiation to form emitted radiation;
using said probe for detecting and mapping said emitted radiation;
mapping the prostate using at least one additional mapping device; and
collecting information from said at least one additional mapping device and said probe, so as to map the prostate.

258. The method of claim 257, wherein said emitted radiation comprises fluorescent X-ray radiation.

259. The method of claim 257, wherein said at least one additional mapping device is selected from the group consisting of an ultrasonic device, a magnetic-resonance-imaging device and a computer tomography device.

260. The method of claim 257, wherein said at least one additional mapping device is endoscopic.

261. The method of claim 257, wherein said endoscopically insetting said probe is into the rectum or the urethra of the subject.

262. The method of claim 257, wherein said detecting said emitted radiation is by a radiation detector which comprises at least one of a stationary detector, a scanning detector, a position-sensitive detector or an array of detectors or a combination thereof.

263. The method of claim 262, wherein said radiation detector is selected from the group consisting of a radiation detector having a single element, a radiation detector having a pixelized array and a radiation detector having an array assembled of a plurality of individual elements.

264. The method of claim 262, wherein said radiation detector comprises at least one of a high energy-resolution solid state detector and a high energy-resolution gaseous detector.

265. The method of claim 264, wherein said high energy-resolution gaseous detector is selected from the group consisting of a gas proportional detector and gas scintillation detector.

266. The method of claim 262, where said high energy-resolution solid state detector is selected from the group consisting of Silicon radiation detector, Germanium radiation detector, Silicon-Lithium-drifted radiation detector, Germanium-Lithium-drifted radiation detector, Mercury Iodide radiation detector and Cadmium-Zinc Telluride radiation detector.

267. The method of claim 266, wherein said high energy-resolution solid state detector is selected from the group consisting of a PIN diode, a surface barrier diode, a drift diode, a micro-strip detector, a drift chamber, a multi-pixel detector and a multi-strip detector.

268. The method of claim 257, wherein said irradiating comprises scanning the prostate so as to excite said chemical element to emit said radiation from a plurality of predetermined angles.

269. The method of claim 257, wherein said detecting said emitted radiation is by scanning the prostate so as to detect said emitted radiation from a plurality of predetermined angles.

270. The method of claim 257, wherein said detecting said emitted radiation is by an arrangement of radiation detectors arranged so as to detect said emitted radiation from a plurality of predetermined angles.

271. The method of claim 257, wherein said chemical element comprises zinc, and wherein the level of zinc is used for detecting a possible cancer in at least a portion of the prostate.

272. The method of claim 257, further comprising introducing at least one radioactive substance into the prostate and measuring the level of said at least one radioactive substance in the prostate.

273. The method of claim 271, further comprising mapping a boundary of said possible cancer in the prostate.

274. The method of claim 273, wherein said mapping said boundary is according to a distribution of said chemical element in at least a region of the prostate being examined.

275. The method of claim 273, wherein said boundary is at least partially determined according to a distribution of different concentrations of said chemical element within at least said region.

276. The method of claim 275, further comprising using said distribution of said different concentrations of said chemical element for staging the cancer.

277. The method of claim 257, wherein said chemical element comprises a chemical element introduced into the prostate for a specific medical procedure, and wherein said mapping the level of said chemical element is used for performing the specific medical procedure on at least a portion of the prostate.

278. The method of claim 277, wherein said specific medical procedure comprises a photodynamic therapy.

279. The method of claim 278, wherein the chemical element is Pd.

280. The method of claim 277, wherein said chemical element is introduced in either a quantitative or a qualitative amount.

281. The method of claim 257, wherein said chemical element to be detected comprises one or more of Zn, Fe, Ca, Br, or Pd.

282. The method of claim 257, wherein said chemical element to be detected comprises Zn.

283. The method of claim 258, wherein said chemical element to be detected emits characteristic fluorescent X-rays according to an identity of said chemical element, and wherein an intensity of said characteristic fluorescent X-rays correlates to a concentration of said chemical element, such that said radiation detector is adapted to detect at least one chemical element according to said characteristic fluorescent X-rays and to measure said intensity.

284. The method of claim 257, wherein said irradiating the prostate is by an irradiation system comprising at least one of a radioactive source, an X-ray tube, a synchrotron light source, an X-ray beam guide connected to an external X-ray source or a miniature plasma X-ray generator.

285. The method of claim 257, wherein said irradiation system is coupled to a monochromatizing element so as to provide a radiation with a substantially accurate energy.

286. The method of claim 285, wherein said monochromatizing element is selected from the group consisting of a crystal monochromator and a plurality of different absorbing films each characterized by a different absorption coefficient.

287. The method of claim 257, further comprising using said probe for performing a biopsy procedure.

288. The method of claim 257, further comprising using said probe for injection of a drug or a contrast agent into the prostate.

289. The method of claim 257, further comprising using said probe for illuminating the prostate with light.

290. The method of claim 257, further comprising a normalizing measurement of said emitted radiation according to a normalizing measurement of a reference element.

291. The system of claim 290, wherein said normalizing is according to an amount of Compton scattered radiation of radiation emitted by said irradiation system

292. The method of claim 257, further comprising collimating and focusing said exciting radiation and said emitted radiation.

293. The method of claim 262, further comprising cooling said radiation detector to have improved energy resolution.

294. The method of claim 293, wherein said cooling said radiation detector is by a thermoelectric cooling system, adapted for being located within said probe.

295. The method of claim 257, further comprising discriminating between radiation emitted by said chemical element being present in the prostate and radiation emitted by chemical elements being present in tissues surrounding said prostate, thereby to map the prostate.

296. The method of claim 257, further collimating said emitted radiation in a manner that radiation emitted by chemical elements being present in tissues other than tissues of the prostate is absorbed.

297. The method of claim 257, further comprising simultaneously detecting said emitted radiation from a plurality of depth inside the prostate.

298. The method of claim 257, further comprising collimating radiation emitted from different depths inside the prostate into different locations of a radiation detector or different radiation detectors.
